(19) United States
(12) Patent Application Publication
(10) Pub. No.: US 2018/0226148 A1
(43) Pub. Date: Aug. 9, 2018

(54) TELEMONITORING AND ANALYSIS SYSTEM

(71) Applicant: Esvyda!, Inc., Campbell, CA (US)

(72) Inventors: Elias Lozano, Campbell, CA (US); Wilson David Jaramillo Romero, Pereira (CO); Paola Bonilla Galindo, Pereira (CO); David Alexander Murillo Gaviria, Pereira (CO)

(21) Appl. No.: 15/889,353

(22) Filed: Feb. 6, 2018

Related U.S. Application Data
(60) Provisional application No. 62/455,570, filed on Feb. 6, 2017.

(51) Int. Cl. G16H 20/60; G16H 80/00

(52) U.S. Cl. CPC G16H 20/60; G16H 80/00

(57) ABSTRACT

Disclosed is a telemonitoring and analysis system and associated methods. A doctor creates a care plan for a patient with a chronic condition, such as diabetes. The care plan can include a medication plan, exercise plan, healthy eating plan, etc., and the care plan is input into a telemonitoring and analysis system. During the care plan, the telemonitoring and analysis system collects data on medical parameters and medical events and analyzes this data to determine an effectiveness of the care plan based on compliance metrics, medical event metrics and information from external sources.

### CROSS-REFERENCE TO RELATED APPLICATIONS

This application claims the benefit of U.S. Provisional Patent Application No. 62/455,570, filed Feb. 6, 2017, which is incorporated by reference herein in its entirety for all purposes. This application is related to U.S. patent application Ser. No. 15/431,646, which is incorporated by reference herein in its entirety for all purposes.

### BACKGROUND

A patient visits a doctor for a medical condition, and the doctor evaluates the patient and makes a diagnosis. The doctor writes up a care plan that includes a medication plan, exercise plan, healthy eating plan, and biometric testing plan. When the patient returns home, in some cases, he attempts to follow the plan but sometimes neglects to follow his care plan perfectly. Moreover, the doctor can have difficulty in assessing the effectiveness of the care plan because there are many factors that can affect whether the plan was effective such as whether the patient took each dose of medication, exercised, or experienced allergies or adverse drug effects. Without knowing the effectiveness of the care plan, it is difficult for the doctor to know what adjustments should be made.

### BRIEF DESCRIPTION OF THE DRAWINGS

FIG. 1A is a system diagram that illustrates components of a telemonitoring and analysis system (referred to as “TAS” in the figures), consistent with various embodiments.

FIG. 1B is a system diagram that illustrates the interaction between components of a telemonitoring and analysis system, consistent with various embodiments.

FIGS. 2A-2B are flow diagrams that illustrate a process to generate a care plan and to determine the effectiveness of the care plan, consistent with various embodiments.

FIGS. 3A-3B are flow diagrams that illustrate a process to engage users as consumers into the telemonitoring and analysis system, consistent with various embodiments.

FIGS. 4A-4B are flow diagrams that illustrate a process to engage patients into the telemonitoring and analysis system under care provider supervision, consistent with various embodiments.

FIGS. 5A-5D are flow diagrams that illustrate a process of creating a database for a care plan, consistent with various embodiments.

FIG. 6 is a flow diagram that illustrates a process of creating reconciliation notes from a medication history in a telemonitoring and analysis system, consistent with various embodiments.

FIGS. 7A-7B are flow diagrams that illustrate a process of documenting medical events resulting in alerts into the telemonitoring and analysis system, consistent with various embodiments.

FIG. 8 is a flow diagram that illustrates a process of synchronizing data between a telemonitoring and analysis system, devices and applications, consistent with various embodiments.

FIGS. 9A-9B are flow diagrams that illustrate a process of synchronizing prescription data from a telemonitoring and analysis system to a mobile application, consistent with various embodiments.

FIG. 10 is a flow diagram that illustrates a process of collecting and synchronizing confirmed reminders from a telemonitoring and analysis system to a mobile application, consistent with various embodiments.

FIGS. 11A-11B are flow diagrams that illustrate a process for a telemonitoring and analysis system to collect medical parameter data from various devices, consistent with various embodiments.

FIG. 12 is a flow diagram that illustrates alerts being generated within a telemonitoring and analysis system according to a care plan, consistent with various embodiments.

FIG. 13 is a flow diagram that illustrates a process for classifying alerts within a telemonitoring and analysis system, consistent with various embodiments.

FIGS. 14A, 14B, and 14C are flow diagrams that illustrate a process for processing a user-generated alert within a telemonitoring and analysis system, consistent with various embodiments.

FIGS. 15A-15B are flow diagrams that illustrate a process for processing a caregiver-generated alert within a telemonitoring and analysis system, consistent with various embodiments.

FIG. 16 is a flow diagram that illustrates a process for confirming medication orders prescribed by care providers through the telemonitoring and analysis system, consistent with various embodiments.

FIG. 17 is a flow diagram that illustrates a process for establishing medication adherence and compliance levels using a telemonitoring and analysis system, consistent with various embodiments.

FIGS. 18A-18B are flow diagrams that illustrate a process for creating medical adherence and compliance statistics by the telemonitoring and analysis system, consistent with various embodiments.

FIG. 19 is a flow diagram that illustrates a process for determining effectiveness of a care plan using a telemonitoring and analysis system, consistent with various embodiments.

FIG. 20 is a flow diagram that illustrates a process for determining the variables used in calculating the progress of medical parameters using a telemonitoring and analysis system, consistent with various embodiments.

FIGS. 20A-1, 20A-2, 20A-3, and 20A-4 illustrate an example of data and a calculation of medical parameters metrics during a care plan, consistent with various embodiments.

FIG. 21 is a flow diagram that illustrates a process for determining the variables that will be used in calculating the progress of the patient based on medical events using a telemonitoring and analysis system, consistent with various embodiments.

FIGS. 21A-1, 21A-2, 21A-3, 21A-4, 21A-5, and 21A-6 illustrate examples of data and a calculation of metrics based on medical events and external source parameters, consistent with various embodiments.

FIGS. 22A-22B are flow diagrams that illustrate a process for classifying medical parameters and medical events to calculate effectiveness of a care plan by a telemonitoring and analysis system, consistent with various embodiments.

FIGS. 22A-1, 22A-2, and 22A-3 illustrate examples of data and a total calculation between medical parameters, medical events and external sources to determine effective of the care plan, consistent with various embodiments.

FIGS. 23A-23B are flow diagrams that illustrate a process for determining the effectiveness of a care plan by a telemonitoring and analysis system, consistent with various embodiments.

FIG. 24 illustrates an example of an interface that can be used for medication management of users in a telemonitoring and analysis system, consistent with various embodiments.

FIG. 25 illustrates examples of medication care plan module interfaces of the telemonitoring and analysis system, consistent with various embodiments.

FIG. 26 illustrates an example of a medication module interface of active and inactive medications prescribed to treat health conditions and a medication compliance score of a user, consistent with various embodiments.

FIG. 27 illustrates an example of a medication order consultation module interface for the evaluation of the diagnosis of users, consistent with various embodiments.

FIG. 28 illustrates an example medication reconciliation module interface of a medication history of a user, consistent with various embodiments.

FIGS. 29A-29B illustrate examples of alerts and notifications module interfaces that notifies users of generated events, consistent with various embodiments.

FIG. 30 illustrates an example of a mood module interface that allows users to report current status against medications prescribed, consistent with various embodiments.

FIGS. 31A-31B illustrate examples of events report module interfaces that allow users or care providers to report any medical event related to the care plan, consistent with various embodiments.

FIG. 32 illustrates an example alerts and notifications module interface with the vital sign alerts generated by users engaged in a care plan, consistent with various embodiments.

FIG. 33 illustrates an example of an engagement module interface that shows the medication adherence metrics during the progression of the care plan assigned to a user, consistent with various embodiments.

FIG. 34 illustrates an example care plan program module interface that shows the effectiveness based on medical parameters and medical events, consistent with various embodiments.

FIG. 35 is a system block diagram illustrating a computer system in which at least some operations described herein can be implemented, consistent with various embodiments.

### DETAILED DESCRIPTION

Introduced here is technology related to a telemonitoring and analysis system, which is a system for remotely monitoring patients who are not at the same location as a health care provider and for analyzing the effectiveness of a care plan of a patient, including the effectiveness of the medications included in the care plan.

A person with a health care condition can be evaluated by a care provider. The care provider can diagnose the patient and develop a care plan that includes a medication plan, exercise plan, nutrition plan, education plan, medical review plan, and biometric testing plan, and the care plan can be input into a database of a telemonitoring and analysis system. After the care provider prescribes the patient's medications and the medications are dispensed and confirmed in the system, the care plan is activated. When the patient obtains the medications, the patient starts taking the medications and vital sign readings according to the care plan.

Throughout the course of the care plan, events such as taking medication, problems, alerts, and improvements associated with his/her condition can be reported to the care provider and/or caregiver via the telemonitoring and analysis system. Various devices such as a mobile application on the patient's mobile device and medical devices such as a drug dispenser device can be used to provide the updates to the telemonitoring and analysis system. In an example, the telemonitoring and analysis system can determine that it is time for the patient to take a medication. In response, the telemonitoring and analysis system can send a message to the patient's mobile device, which triggers a care plan application running on the mobile device to display an alert that it is time to take a particular medication. The patient can take the medication and tap an icon on the mobile device to indicate that he/she took the medication. A few hours later, the patient can develop a headache and can report such symptom in the telemonitoring and analysis system via the mobile application. The updates provided by the patient and/or the medical device can be reviewed by the care provider and/or caregiver. Upon reviewing the updates provided by the mobile application and various devices, the care provider supports and gives recommendations to the patient. When the care plan is complete, or sometimes during the care plan (e.g., at the end a period, after an alert is generated) the system notifies the care provider, and the care provider can make adjustments accordingly.

The telemonitoring and analysis system can analyze data taken during the care plan to determine the effectiveness of the medication and the care plan. The data can include medical parameter data relating to medical parameters (e.g., when the patient took medications, how much medication was taken, vital signs taken, biometric information, times the vital signs were taken) and medical event data (e.g., moods; survey information; and events such as symptoms, emergency room visits, allergies, Adverse Drug Effects). In some embodiments, the telemonitoring and analysis system can simultaneously receive the medication data from a mobile device and a drug dispenser device or other medical device, and compare the data. If the data is conflicting, the telemonitoring and analysis system can select the medication data based on a reliability of the source of information.

To determine the effectiveness of the medication and the care plan, the telemonitoring and analysis system can determine an adherence level for each of the medical parameters using the medical parameter data. More specifically, the telemonitoring and analysis system can compare the medical parameter data for each of the scheduled care plan events with expected medical parameter data for each of the events (e.g., correct medicine taken at correct time). The telemonitoring and analysis system can classify the medical parameter data into scoring classes based on the comparison (e.g., in range, out of range) and assign a weighted value to the medical parameter data for each of the scheduled care plan events, where the weighted value is based on the scoring class (e.g., in range is 0 points, out of range is −10 points). The telemonitoring and analysis system can then average the weighted values to determine an adherence level for each of the medical parameters.

In addition to the medical parameter data, the telemonitoring and analysis system can analyze medical event data relating to medical events occurring during the care plan. The telemonitoring and analysis system can generate one or more medical event scores. Certain categories of medical events have subcategories. To generate a medical event score for those categories having subcategories, the telemonitoring and analysis system categories the medical event data into medical event subcategories, assigns a weight to each of the subcategories, determines a score for each of the medical event subcategories based on the weight and the medical event data, and combines the subcategory scores to generate the medical event score. In some embodiments, there are multiple categories of medical events (e.g., alerts, mood, surveys) and each category can have a separate score. Some medical event data can be received by external sources. The external source data can be categorized, weighted, and used as factors in determining effectiveness of the care plan. In some embodiments, some medical events do not have separate subcategories.

Thereafter, the adherence level for each of the medical parameters and the medical event score are normalized. The telemonitoring and analysis system can calculate an effectiveness of the care plan based on the normalized adherence levels and the normalized medical event score, among other things.

In some embodiments, when the medical parameter data or medical event data indicates an issue (e.g., overdose), an alert is sent to a care provider or caregiver.

In some embodiments, the telemonitoring and analysis system can create a graph displaying the calculated level of the effectiveness of the medication(s) in relationship to the vital sign readings.

In some embodiments, the weight of the categories is modified using a correction factor based on the external sources, expert considerations and analysis of the average, variance and covariance of weights assigned to other individuals with similar demographic characteristics (e.g., age, gender, ethnic origin, race and economic status). The final weight modified of every category can be normalized. The purpose of the calculation of the correction factor is not only to keep tailored category weights given to a specific individual, but also to include the analytics of the weights given to other individuals (e.g., if the category weight given to an individual exceeds the average and variance is close to zero, the correction factor will be higher. On the other hand, if the category weight given to an individual is close to the average value and variance is close to zero, the correction factor will be lower. In all the cases if the variance is high, the correction factor will tend to be shorter.

The embodiments set forth herein represent the necessary information to enable those skilled in the art to practice the embodiments, and illustrate the best mode of practicing the embodiments. Upon reading the current description in light of the accompanying figures, those skilled in the art will understand the concepts of the disclosure and will recognize applications of these concepts that are not particularly addressed here. It should be understood that these concepts and applications fall within the scope of the disclosure and the accompanying claims. Although the terms “mobile application” and “mobile device” are used throughout the specification, applications running on any device (i.e., applications not specifically designed as mobile applications, devices other than mobile devices) are contemplated.

The purpose of terminology used herein is only for describing embodiments and is not intended to limit the scope of the disclosure. Where context permits, words using the singular or plural form may also include the plural or singular form, respectively.

As used herein, unless specifically stated otherwise, terms such as “processing,” “computing,” “calculating,” “determining,” “displaying,” “generating,” or the like, refer to actions and processes of a computer or similar electronic computing device that manipulates and transforms data represented as physical (electronic) quantities within the computer's memory or registers into other data similarly represented as physical quantities within the computer's memory, registers, or other such storage medium, transmission or display devices. As used herein, unless specifically stated otherwise, the term “or” encompasses all possible combinations, except where infeasible. For example, if it is stated that a database can include A or B, then, unless specifically stated otherwise or infeasible, the database can include A, or B, or A and B. As a second example, if it is stated that a database can include A, B, or C, then, unless specifically stated otherwise or infeasible, the database can include A, or B, or C, or A and B, or A and C, or B and C, or A and B and C.

As used herein, terms such as “connected,” “coupled,” or the like, refer to any connection or coupling, either direct or indirect, between two or more elements. The coupling or connection between the elements can be physical, logical or a combination thereof. References in this description to “an embodiment,” “one embodiment,” or the like, mean that the particular feature, function, structure or characteristic being described is included in at least one embodiment of the present disclosure. Occurrences of such phrases in this specification do not necessarily all refer to the same embodiment. On the other hand, the embodiments referred to also are not necessarily mutually exclusive.

As used herein, terms such as “cause” and variations thereof refer to either direct causation or indirect causation. For example, a computer system can “cause” an action by sending a message to a second computer system that commands, requests or prompts the second computer system to perform the action. Any number of intermediary devices may examine and/or relay the message during this process. In this regard, a device can “cause” an action even though it may not be known to the device whether the action will ultimately be executed.

Note that in this description, any references to sending or transmitting a message, signal, etc. to another device (recipient device) means that the message is sent with the intention that its information content ultimately be delivered to the recipient device; hence, such references do not mean that the message must be sent directly to the recipient device. That is, unless stated otherwise, there can be one or more intermediary entities that receive and forward the message/signal, either “as is” or in modified form, prior to its delivery to the recipient device. This clarification also applies to any references herein to receiving a message/signal from another device; i.e., direct point-to-point communication is not required unless stated otherwise herein.

Advantages, components and features of the disclosed technology will be set forth in the description and detailed in the following figures. Some challenges overcome by the current disclosure include collecting and analyzing medical parameter data, medical event date, and external source data to determine an effectiveness of a care plan.

Some embodiments of the technology presented here allow for efficient care coordination methods, patient engagement policies, vital sign analytics, and care plan analytics. The health data flow components of the current disclosure are intended to be and generally are in compliance with health regulations and policies.

Some embodiments of the present technology involve a telemonitoring and analysis system, which can integrate all the services and functions required to provide the telemonitoring and analysis service covered by the health data flow. A telemonitoring and analysis system can include some or all of the components described in the current disclosure.

Some embodiments of a telemonitoring and analysis system include web application software that supports a user interface for administrating the functions and services of the telemonitoring and analysis system. The user interface can be designed to address issues of health or technological literacy.

Some embodiments of a telemonitoring and analysis system include an application running at a mobile device that wirelessly communicates with medical devices, such as to collect biometric data (e.g., vital signs) obtained by the medical devices. The application running at the mobile device can be a medical care plan application, among others, and the telemonitoring and analysis system can include the mobile device and the application running at the mobile device. Some embodiments of a telemonitoring and analysis system include hardware components that communicate via a corporate network, and does not include hardware components outside of the corporate network. For example, a telemonitoring and analysis system may be comprised of one or more servers and associated storage, where the servers and storage are owned or managed by a single entity and that communicate with each other via a corporate network of the entity. The mobile device can communicate via any of various wireless technologies, such as via cellular technologies (e.g., GPRS, 3G, 4G), WiFi (IEEE 802.11), Bluetooth, Bluetooth Low Energy (BLE), zigbee, Zwave, GPRS, Near Field Communications (NFC), ANT, ANT+, etc. The mobile device can use an abstract communication driver that supports multiple protocols or any other wireless protocols needed to process health or other data.

A telemonitoring and analysis system can be coupled with online Electronic Health Record (HER) systems and Electronic Data Interchange (EDI) platforms that provide communication with health insurance providers and pharmacy systems. The telemonitoring and analysis system can also be connected with notification suppliers system for sending messages, alerts, audio or video conferencing communication, sending reminders to improve care treatments or reduce communication problems between patients and medical staff, etc.

A telemonitoring and analysis system can include handling patient fragmented information through the use of standard protocols and Application Programming Interfaces (APIs) to integrate the following: synchronization of biometric readings between a mobile application and wireless medical devices, clinical data exchange process with any EHR system, billing claims with health insurance systems and e-prescriptions with the pharmacies, etc.

A telemonitoring and analysis system can enable a health care provider to enroll patients to provide them with telemonitoring and analysis services, to enroll medical staff members to support telemonitoring and analysis services, to enroll caregivers or other care team members to assist with the patient's treatment at home or outside of a hospital/clinic, etc. A telemonitoring and analysis system can assign a unique identifier to enable consolidation of patient clinical and biometric data with the patient's records. To help ensure secure communications between various components of a telemonitoring and analysis system, examples of components including a mobile application, web site, web application, server, etc., the components can obtain a security token to enable secure communication between components of the telemonitoring and analysis system. For example, a mobile application can securely obtain biometric data from a wireless medical device, debug the data, and synchronize the data with other components of a telemonitoring and analysis system.

Reference to various health data flows practiced by a telemonitoring and analysis system will now be made in following embodiments, workflows, data flows and examples, some of which are illustrated in the associated figures. A number of specific details are set forth in order to provide a thorough understanding of the disclosed technology. However, the described health data flow may be practiced without these specific details. Some data flows, methods, procedures, networks or algorithms have been described in general terms so as not unnecessarily confuse aspects of the embodiments.

The disclosed technology describes some embodiments of an “optimized data flow” that integrates wireless medical devices, health care providers, caregivers, medical staff, patients suffering chronic conditions including metabolic syndrome, Electronic Data Interchange (EDI) platforms for data interchange with Insurance providers and pharmacies, Electronic Health Record (EHR) systems, third party notification systems with a web/mobile application for providing telemonitoring of biometrics and collecting the required health data of the patient needed to provide a custom care plan, audio and video communication for constant interaction between patient and health care providers, and efficient billing process for the health care provider doing the telemonitoring.

Some embodiments of the disclosed technology involve a telemonitoring and analysis system that integrates all the services and functions required to provide the telemonitoring and analysis service covered by the health data flow. The patient can apply to be enrolled into a telemonitoring and analysis service under the supervision of the health care provider, receive a unique patient identifier, and the telemonitoring and analysis system can synchronize the patient clinical information.

Using standard protocols and APIs, the telemonitoring and analysis system can integrate efficiently with EHR systems, insurance health plan systems, and pharmacies, to collect patient health data. A telemonitoring and analysis system can provide an API for synchronizing biometric data with mobile applications. Some wireless medical devices can synchronize biometric data acquired by the medical devices with a telemonitoring and analysis system, such as by communicating with a mobile application running at a patient's mobile device. In some embodiments, the patient's mobile device is part of the telemonitoring and analysis system, and in other embodiments, the patient's mobile device sends the biometric data to the telemonitoring and analysis system for synchronization.

In some embodiments, medical staff perform all the functions associated with establishing a patient care plan, such as setting patient biometric parameters, and performing analytics of data acquired by the telemonitoring and analysis system.

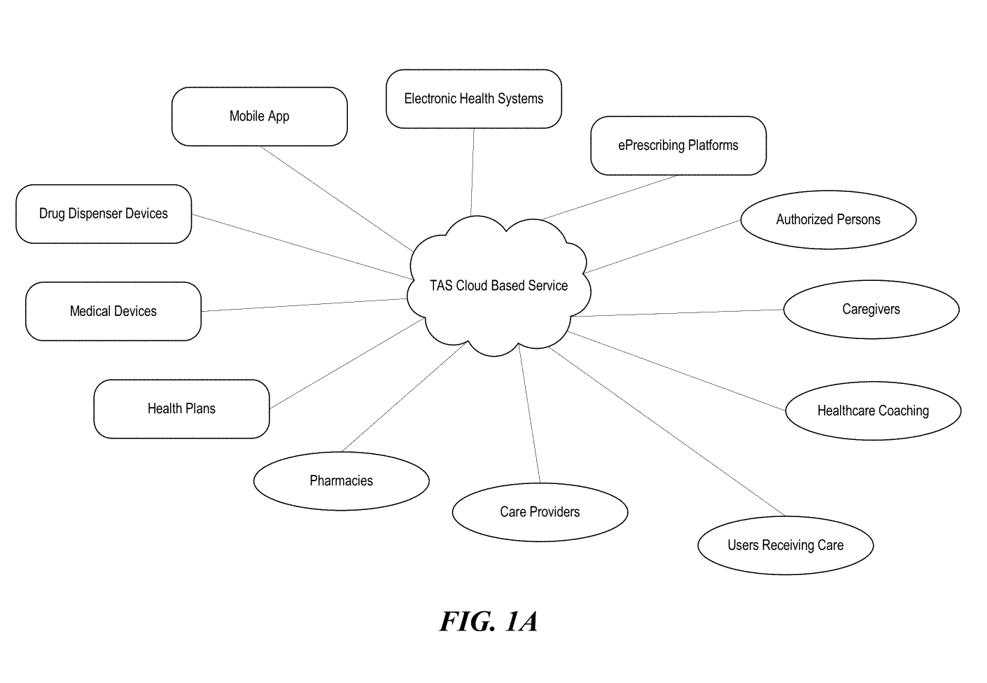

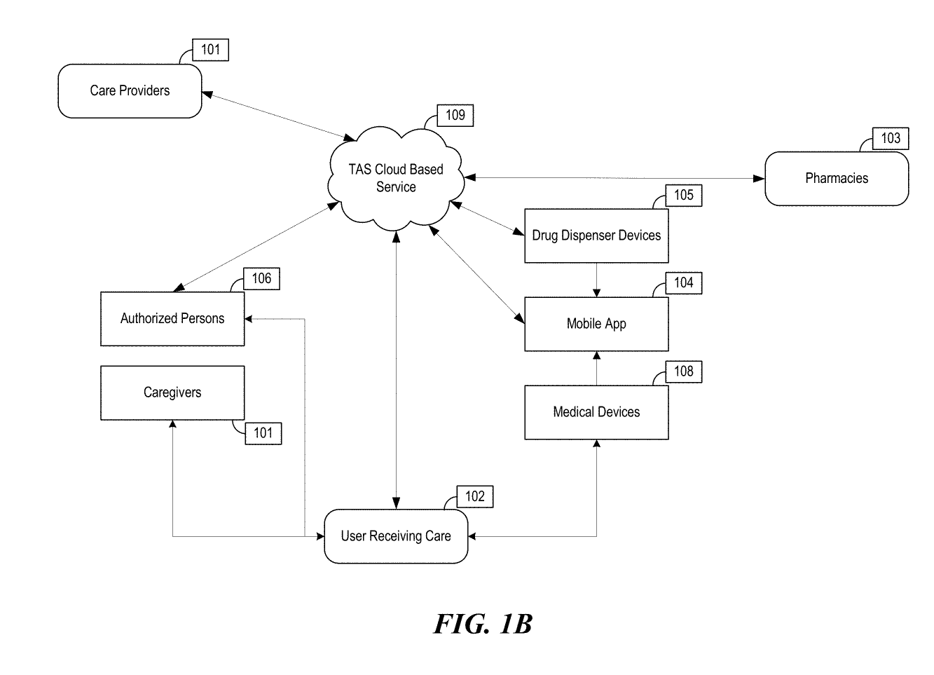

FIG. 1A is a system diagram that illustrates components of a telemonitoring and analysis system and interactions between the components. The system can support care providers and others to enhance the managing of care plan prescriptions by facilitating the administration and the communication between the stakeholders (e.g., caregivers, care providers, patients) making pertinent information available to all users. Users can refer to any user such as care providers (person who provides healthcare services to consumers or patients), pharmacies, caregivers (entities or persons who provide or support patients), patients (individuals who are under care provider supervision), or consumers (persons who self-monitor vitals and medications). In addition, the telemonitoring and analysis system can perform healthcare coaching. For example, users can request an authorized person to assist with medication management and Activities of Daily Living (ADL).

FIG. 1B is a system diagram that illustrates the interaction between components of a telemonitoring and analysis system, including connections between the stakeholders and the devices that will collect the information related to the care plan. Care providers (101) can assign a care plan to a user who will receive care. The telemonitoring and analysis system can allow the user to access his/her progress regarding medications, vital sign goals, events and ADLs planned. Such information can be monitored by care providers, caregivers or authorized persons including members from a pharmacy (103) or a health care coach. The pharmacy can receive information about medication prescriptions and care plan information associated with the user in care either to provide support or administer medications directly to the user (102); authorized persons (106) and caregivers (107) will have access to the user profile to check the care plan progress and to give support if needed. In addition, all the individuals involved in the telemonitoring and analysis system can communicate any event, solution, or changes in case the user in care needs the attention. The devices involved in the telemonitoring and analysis system transmit information collected when users in care take a medication or a vital sign reading. These devices can include a drug dispenser device (105) and medical devices that generate vital sign metrics (108) a mobile application (104) to the system. The information can be sent to the telemonitoring and analysis system, allowing users (including care providers and patients) to check the progress and status of the medication and vital sign conditions as the user continues with the care plan.

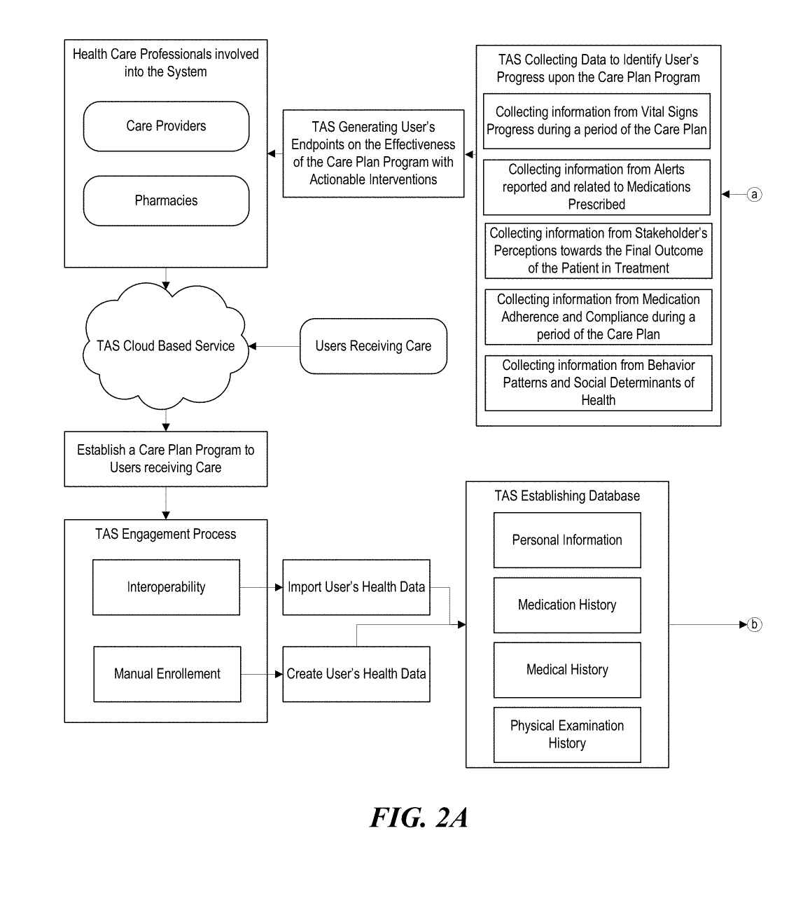

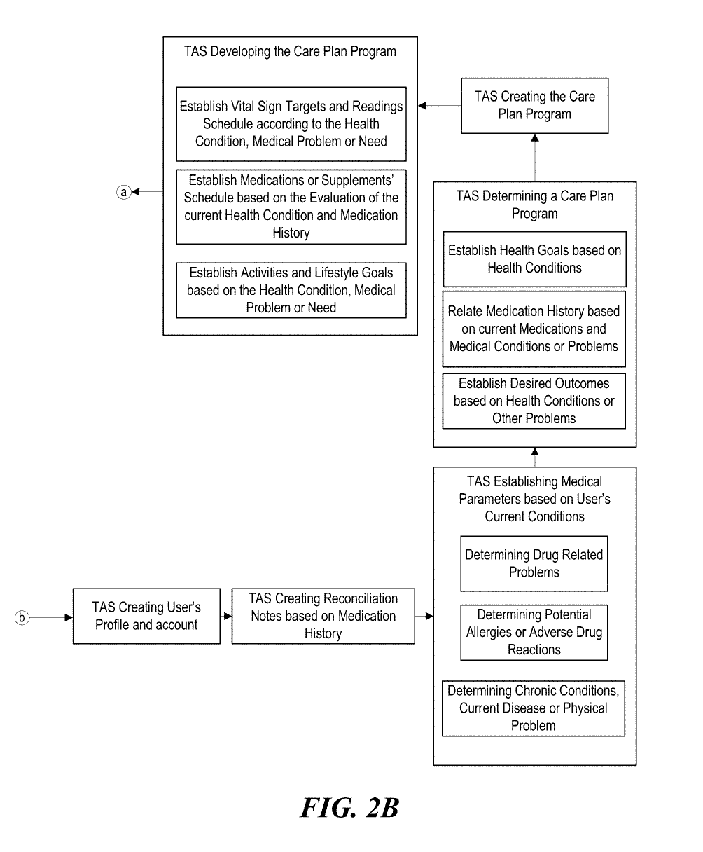

FIGS. 2A-2B are flow diagrams that illustrate a process to generate a care plan and to determine an effectiveness of the care plan.

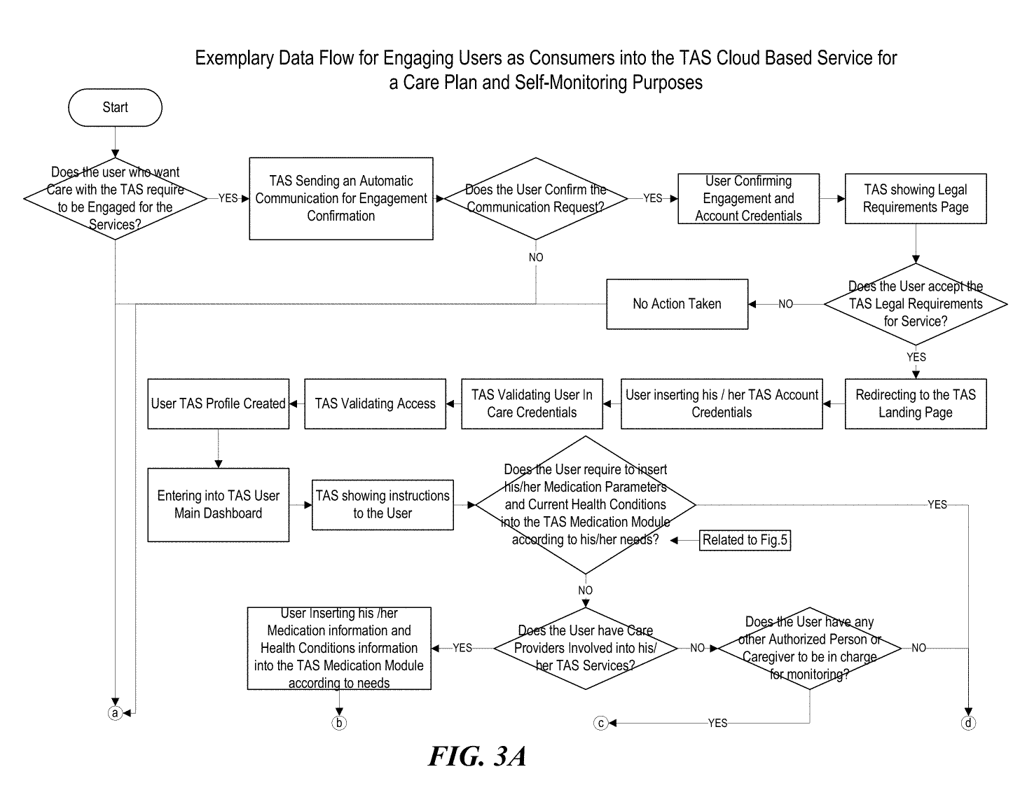

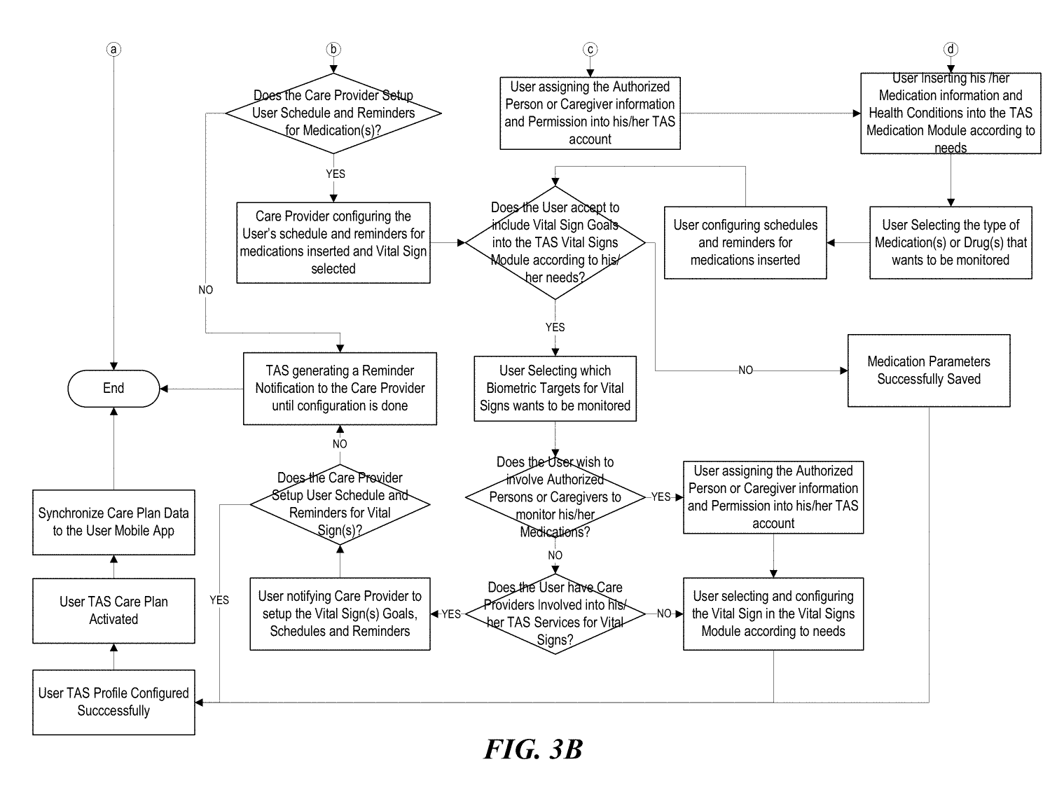

FIGS. 3A-3B are flow diagrams that illustrate a process to engage users as consumers into the telemonitoring and analysis system.

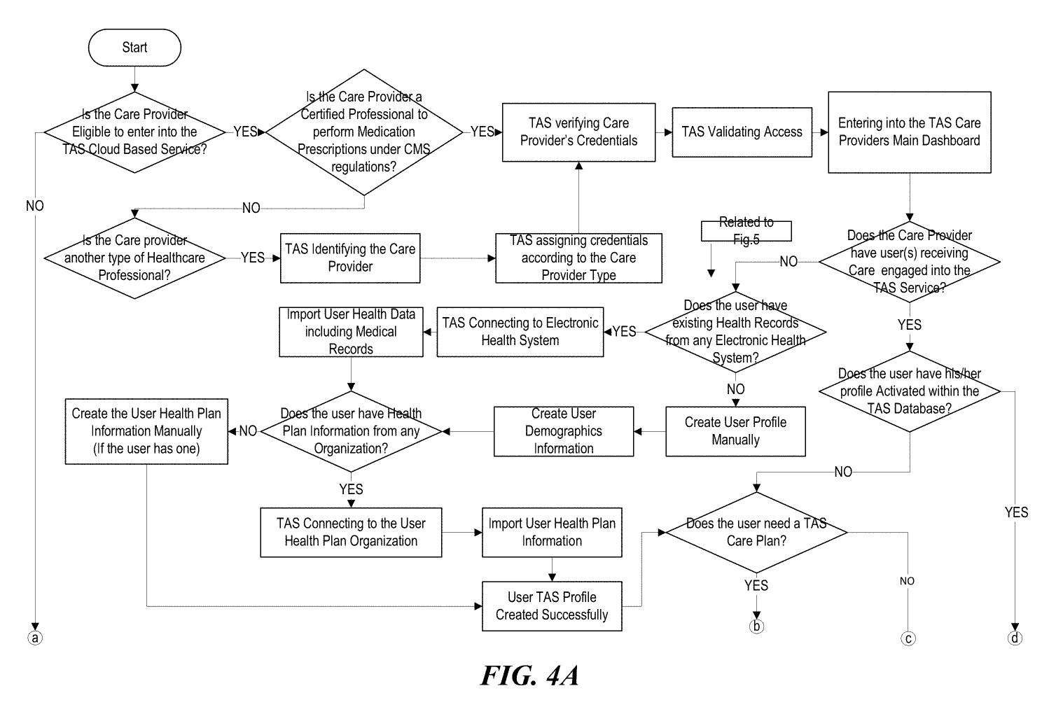

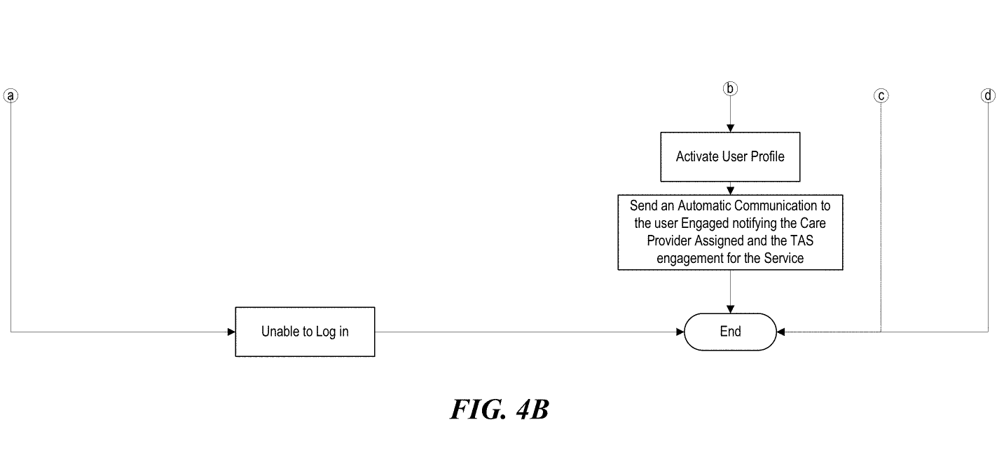

FIGS. 4A-4B are flow diagrams that illustrate a process to engage patients into the telemonitoring and analysis system under care provider supervision.

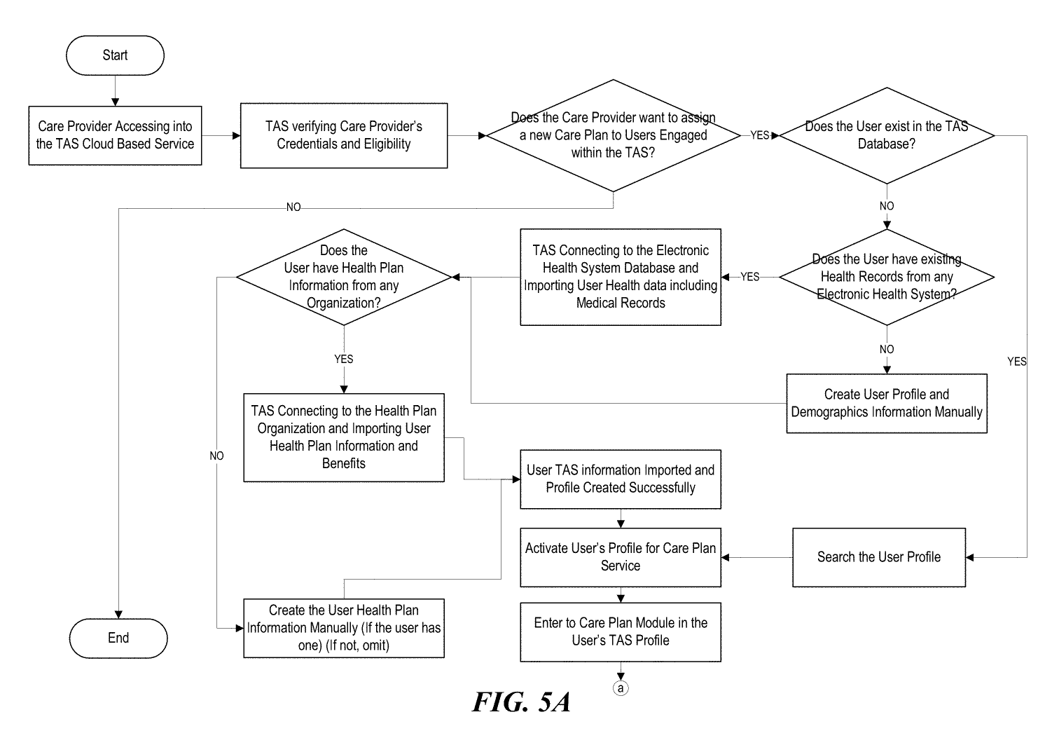

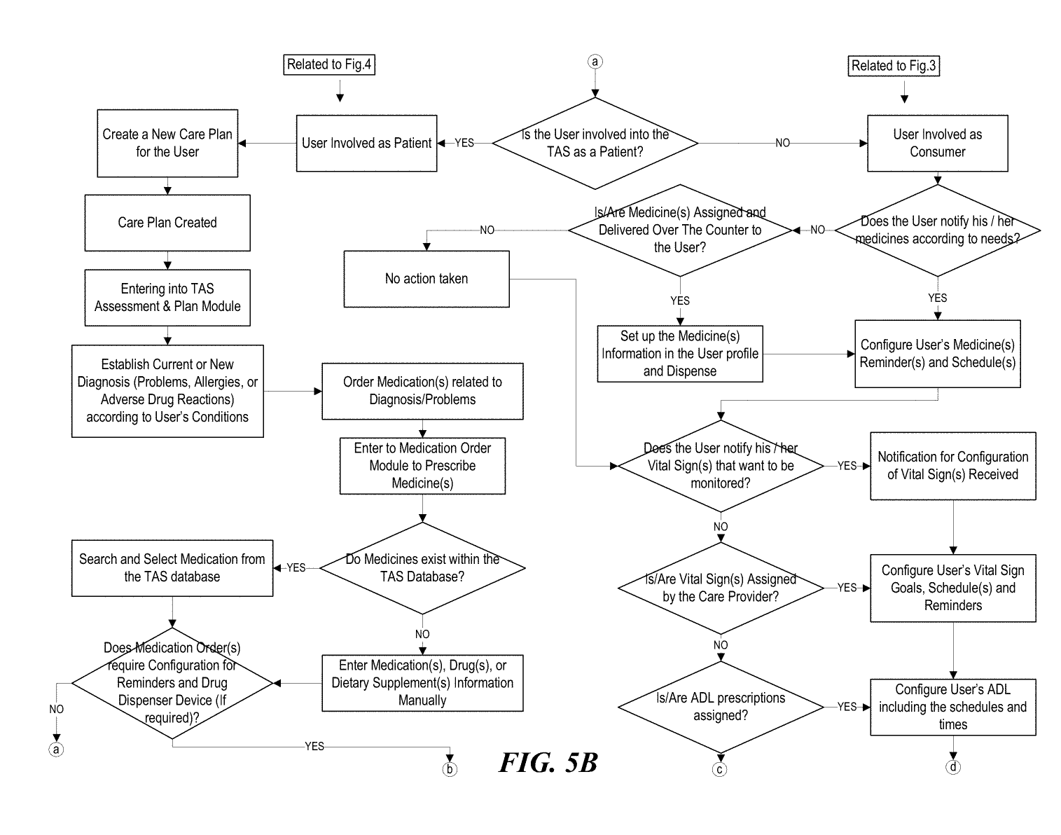

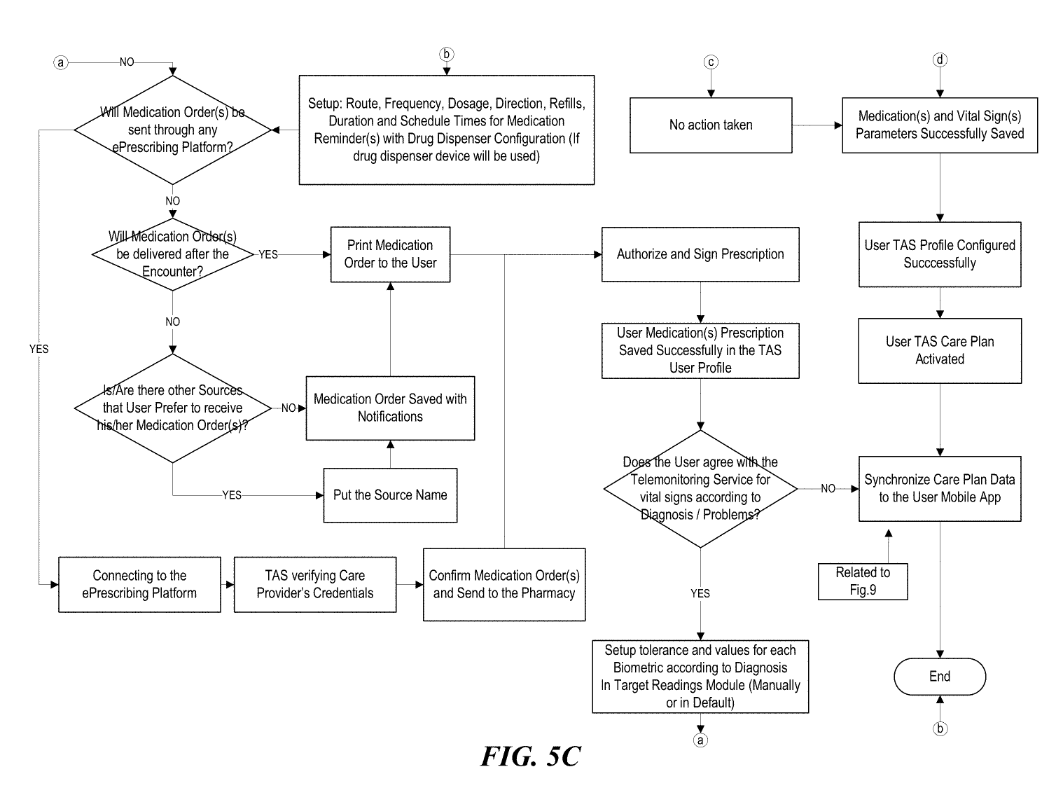

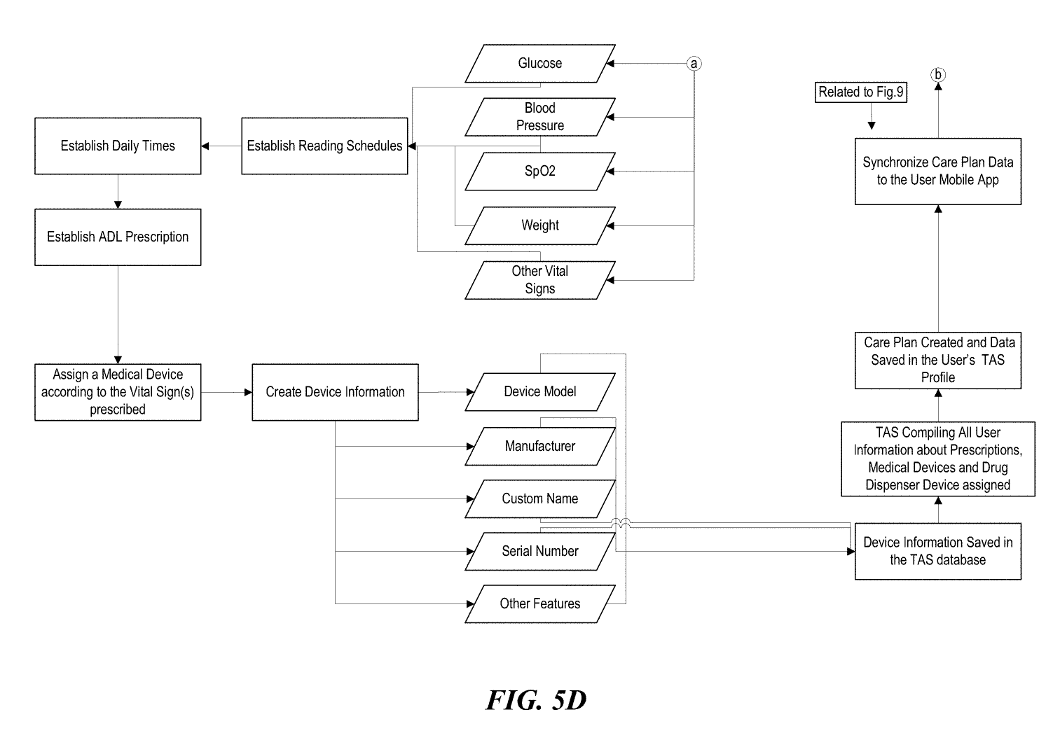

FIGS. 5A-5C are flow diagrams that illustrate a process of creating a database for a care plan.

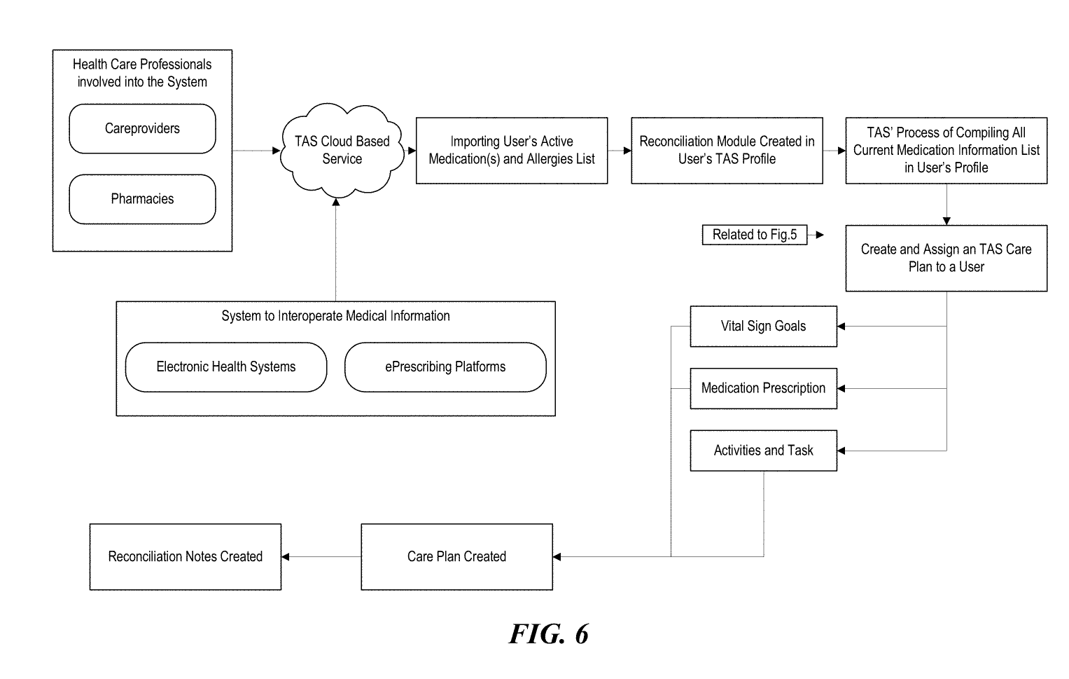

FIG. 6 is a flow diagram that illustrates a process of creating reconciliation notes from a medication history in a telemonitoring and analysis system.

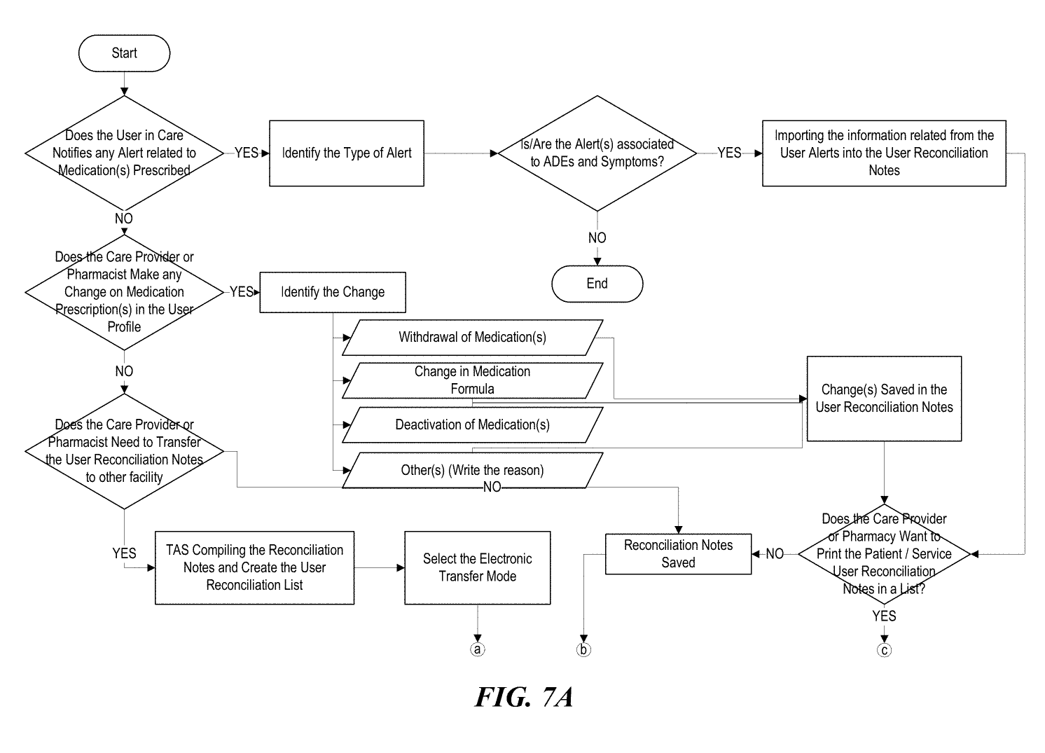

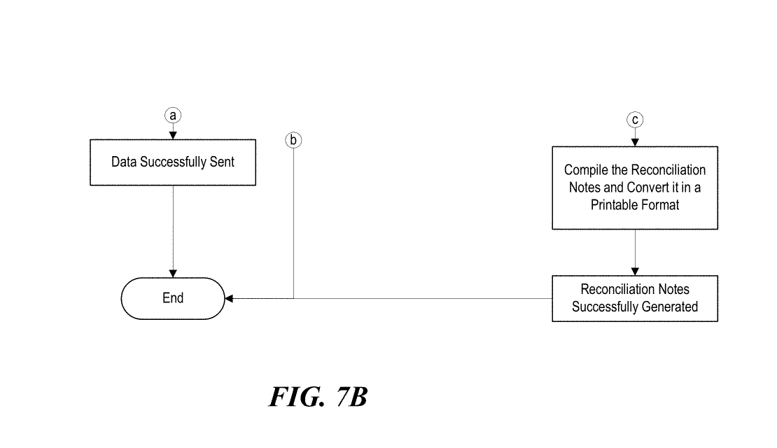

FIGS. 7A-7B are flow diagrams that illustrate a process of documenting medical events resulting in alerts into the telemonitoring and analysis system.

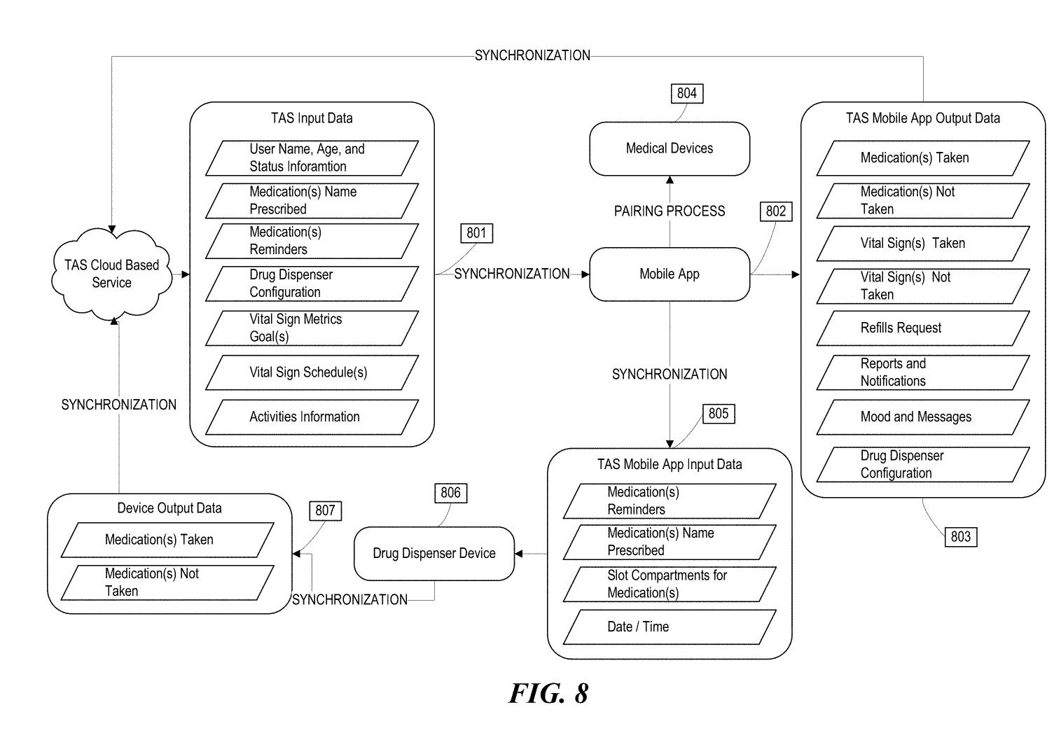

FIG. 8 is a flow diagram that illustrates a process of synchronizing data between a telemonitoring and analysis system, devices, and applications. Data is synchronized from the telemonitoring and analysis system to different channels involved with a care plan. The channels can include mobile devices, mobile applications and medical devices such as a drug dispenser device. The input data that is created within the telemonitoring and analysis system when the care plan is assigned is synchronized to the mobile application associated with the user. The data can include user demographics, names of prescribed medications, medication reminders, drug dispenser configurations (if the user required it), vital sign metrics goals, vital signs schedules, and activities information (e.g., ADL) (801). Such data can refer to the medical parameters inserted into the care plan during a consultation, evaluation or diagnosis of the of the patient, as shown in FIGS. 5A-D.

The care plan includes medical parameters (e.g., medication doses, when to take medication, biometric information, when to take vital signs). Once the medical parameters are created, the telemonitoring and analysis system will synchronize the medical parameters to the mobile application (802) that in turn allows users to check new prescriptions associated with the care plan. In some embodiments, users prefer to monitor their medication intake without using a drug dispenser device. If so, the telemonitoring and analysis system will send reminders via the mobile application reminding the user to take the medicine prescribed or to take a vital sign. Once a medication or vital sign reminder is generated on the mobile application, output data (i.e., medical parameter data) of various data parameters according to the scheduled time established in the user's care plan will be synchronized to the telemonitoring and analysis system. Examples of medical parameter data include medications and vital signs taken/not taken, medications taken in range/out of range, refills requests, reports/notifications of events, drug dispenser status configuration and messages (803). The medical parameter data is synchronized and received into the telemonitoring and analysis system to be shared by the care providers, authorized persons, and other stakeholders. The mobile application can also connect to medical devices (804) that generate biometric information electronically or in default, manually, when the biometric information cannot be shared directly with the mobile application. In some embodiments, when users have an assigned drug dispense device (806), the telemonitoring and analysis system can electronically notify the user when to take medications. Drug dispenser device (806) can further generate accurate information for the telemonitoring and analysis system that is used to determine user adherence levels and compliance during the care plan (or other period of time). Thus, the drug dispenser device can communicate wirelessly with the system and in doing so can send medication adherence information (i.e., medication taken and not taken, in range, out of range readings) to the telemonitoring and analysis system (807). In addition, the mobile application can synchronize data such as medical parameter data and prescriptions with the medical devices. Additional data that can be synchronized between devices includes medication names, reminders configured by care providers, proper medical device configurations for the care plan, date and time for the medications to be prescribed or for vitals to be taken (805).

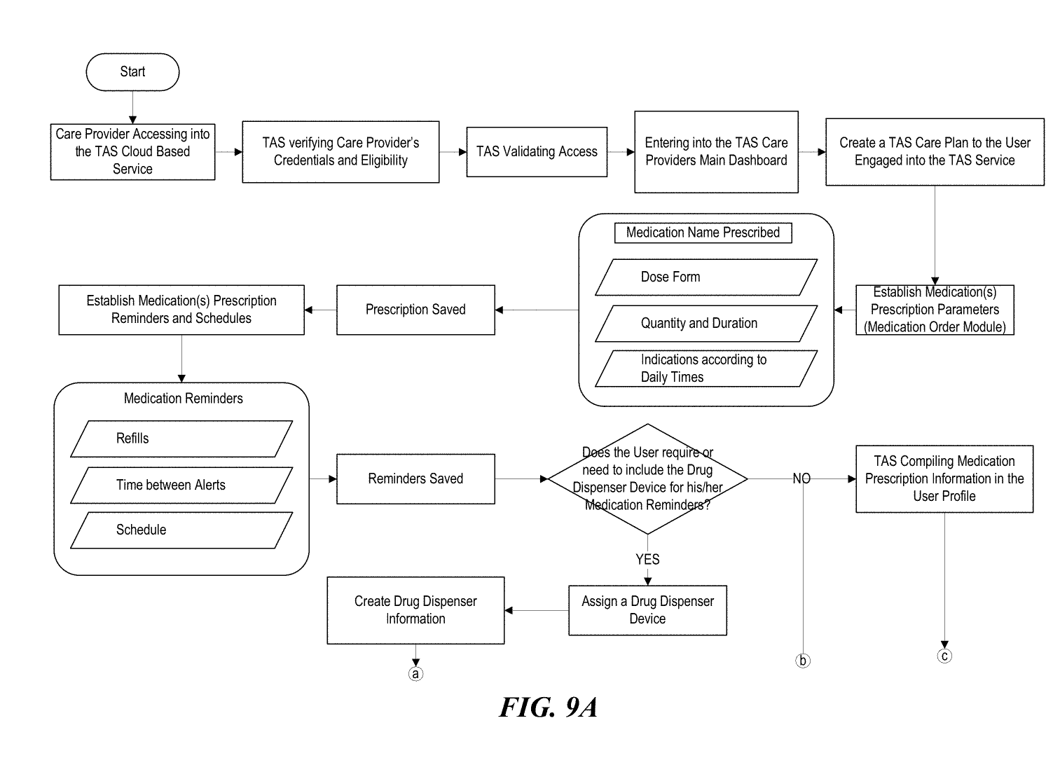

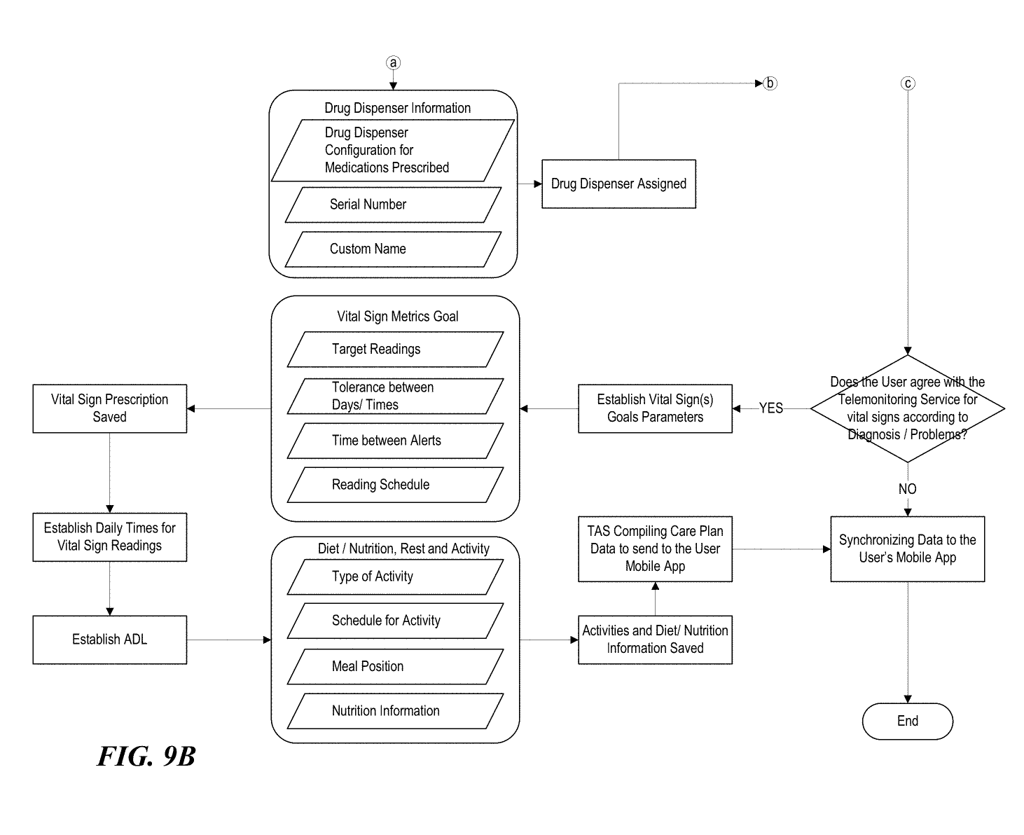

FIGS. 9A-9B are flow diagrams that illustrate a process of synchronizing prescription data from a telemonitoring and analysis system to a mobile application.

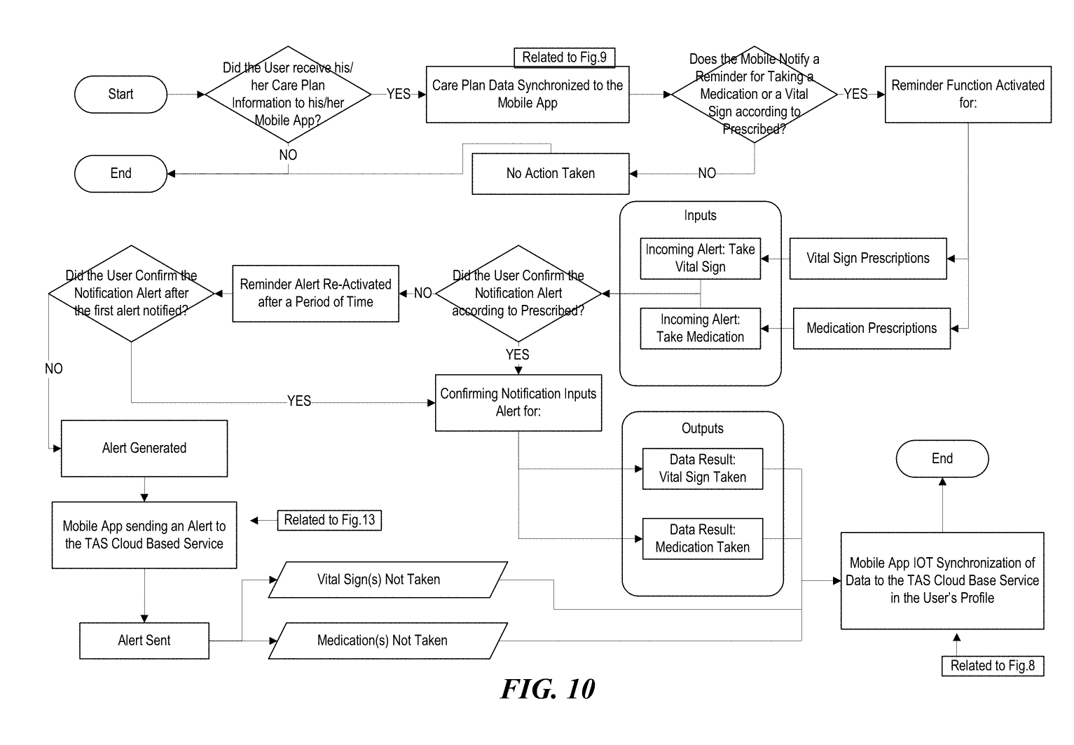

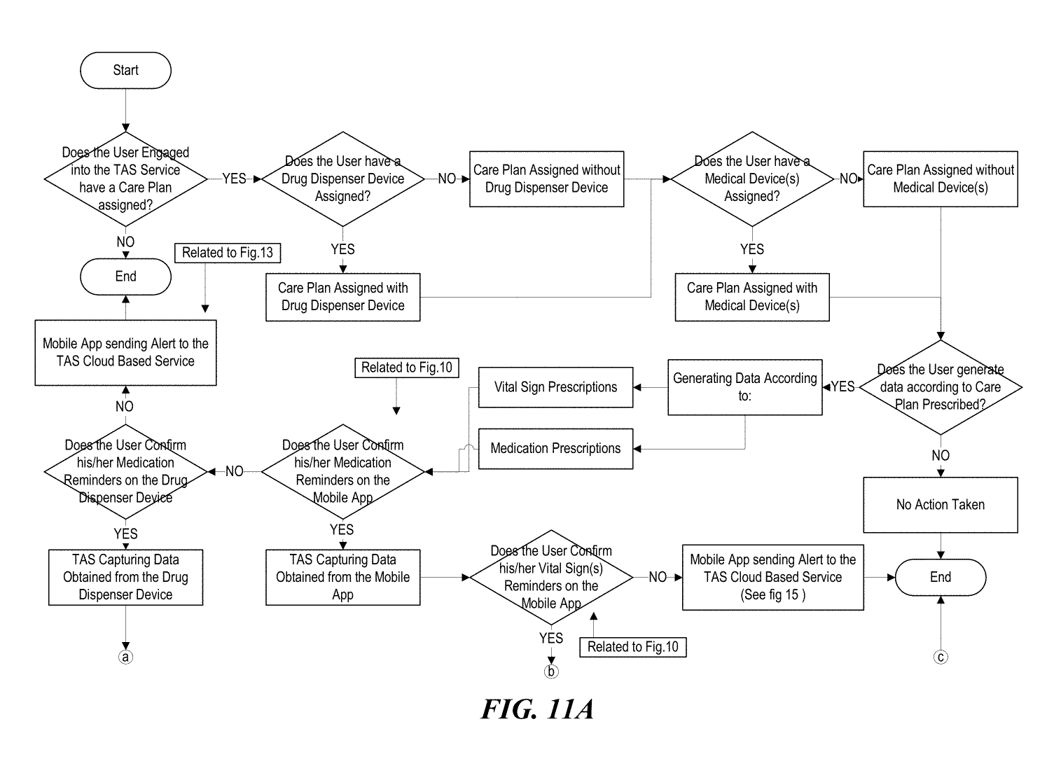

FIG. 10 is a flow diagram that illustrates a process of collecting and synchronizing confirmed reminders from a telemonitoring and analysis system to a mobile application.

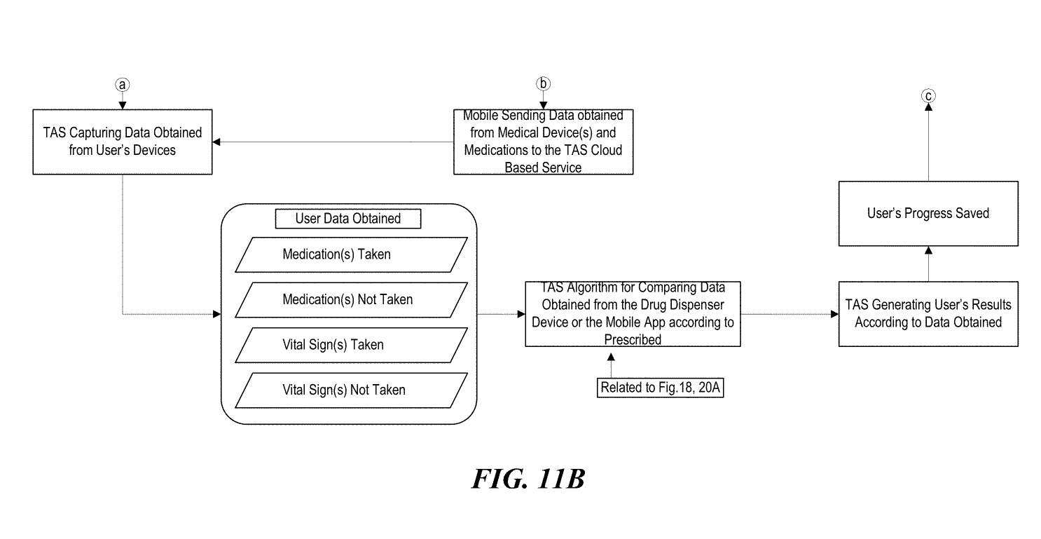

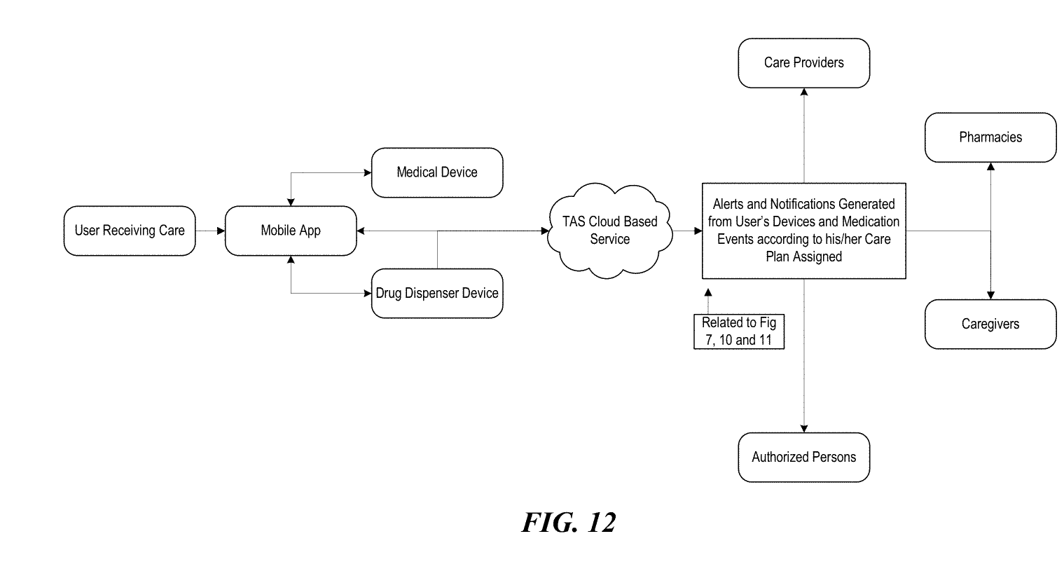

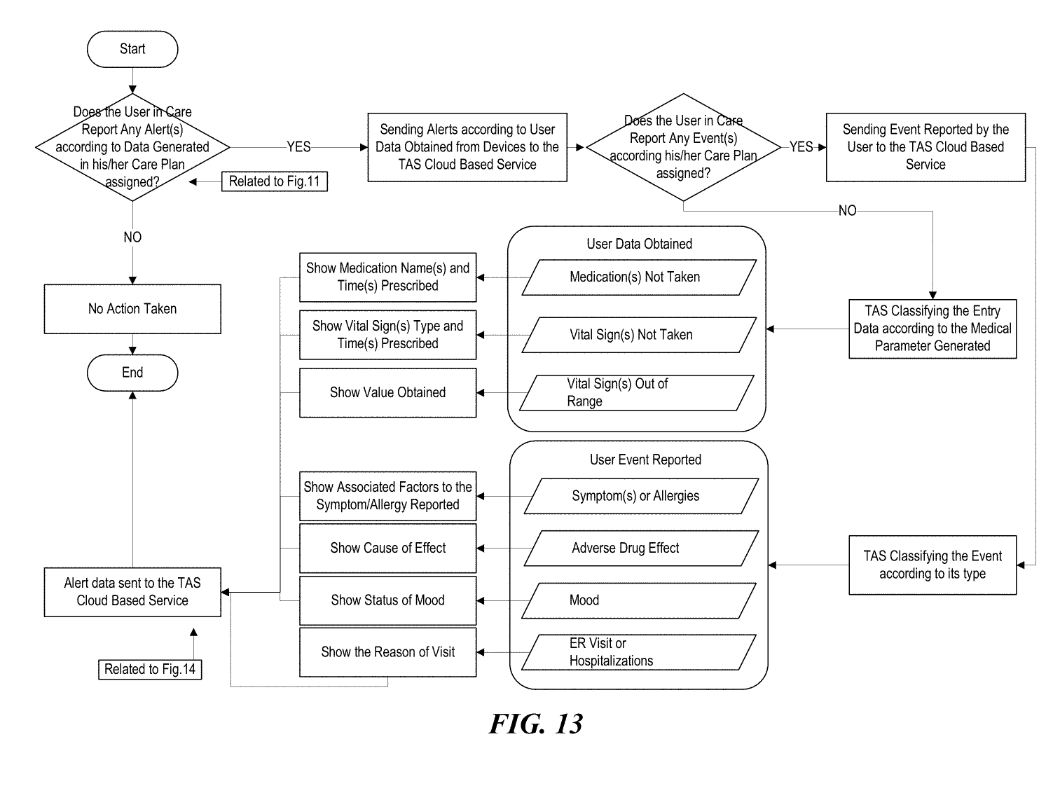

FIGS. 11A-11B are flow diagrams that illustrate a process for a telemonitoring and analysis system to collect medical parameter data from various devices.

FIG. 12 is a flow diagram that illustrates alerts being generated within a telemonitoring and analysis system according to a care plan.

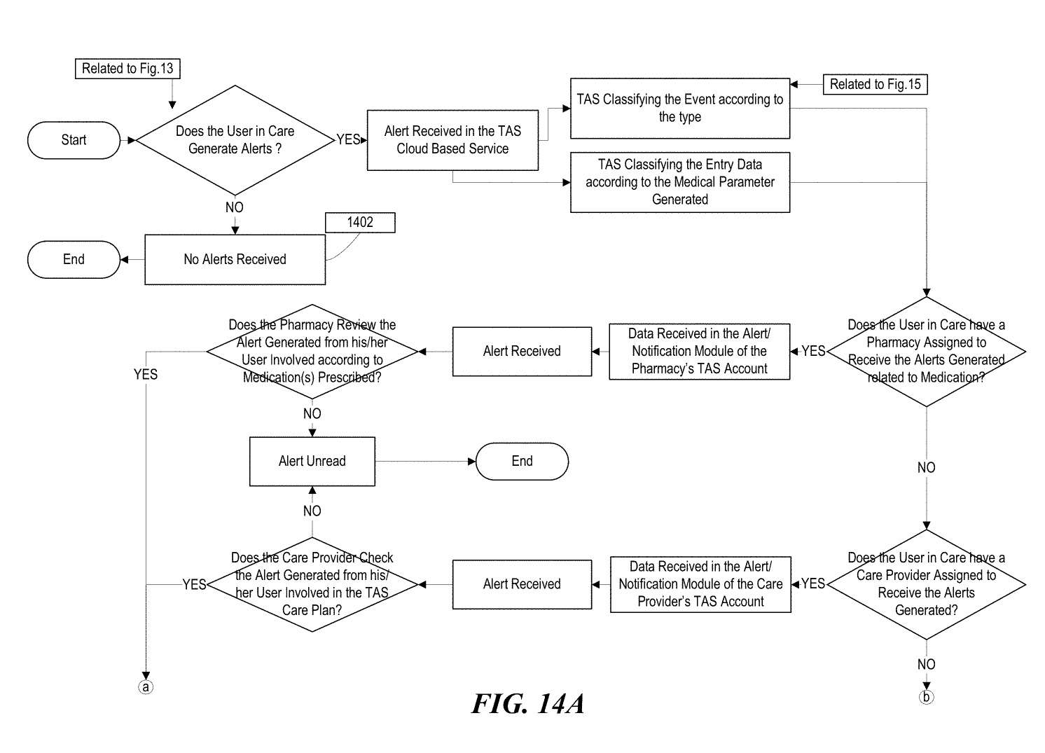

FIG. 13 is a flow diagram that illustrates a process for classifying alerts within a telemonitoring and analysis system.

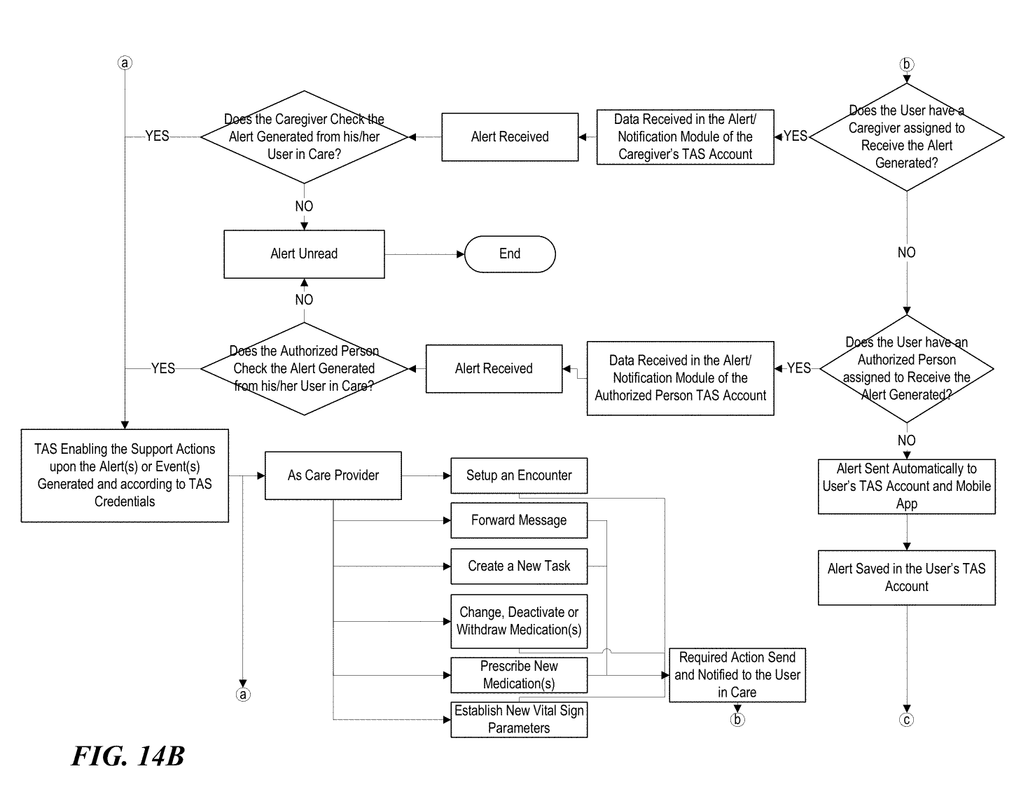

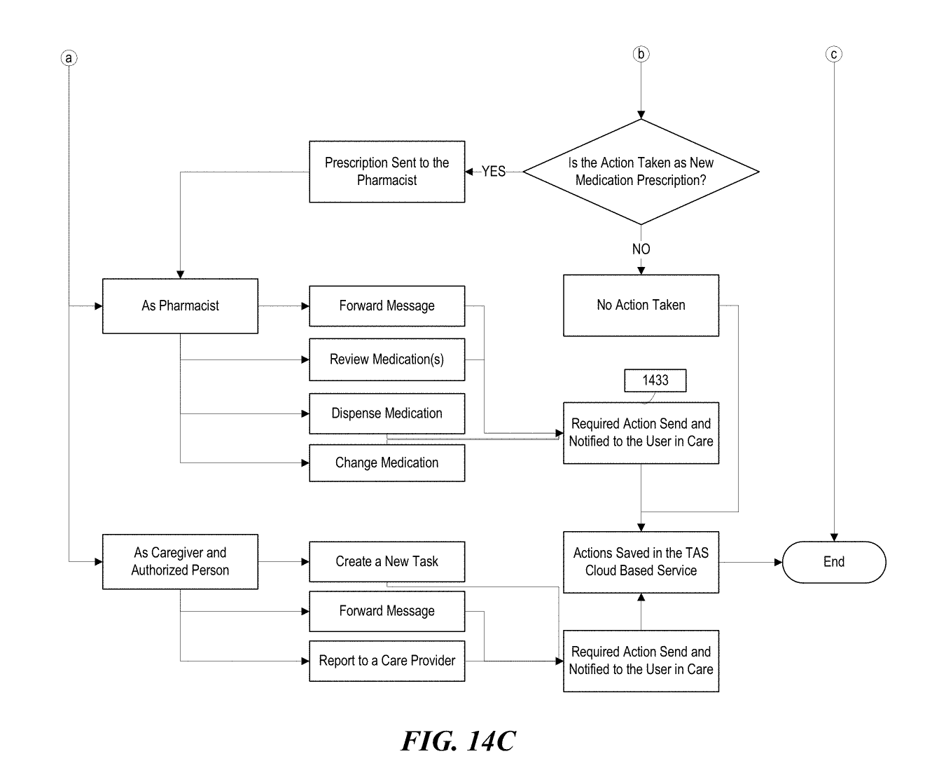

FIGS. 14A, 14B, and 14C are flow diagrams that illustrate a process for processing a user-generated alert within a telemonitoring and analysis system.

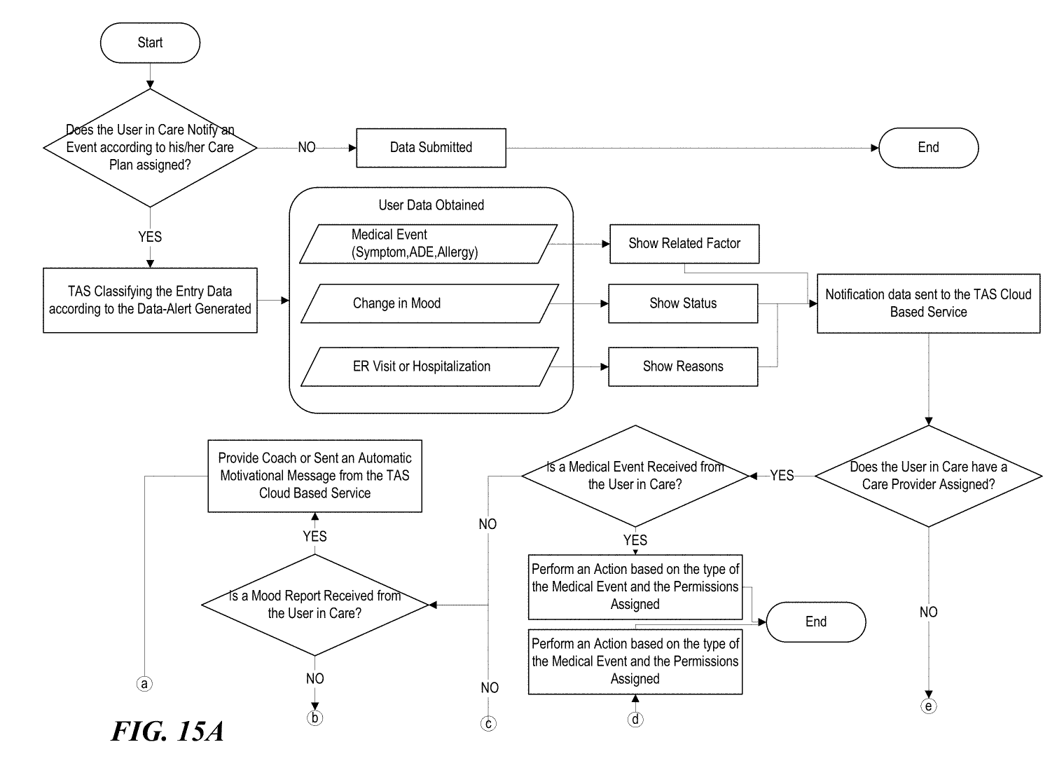

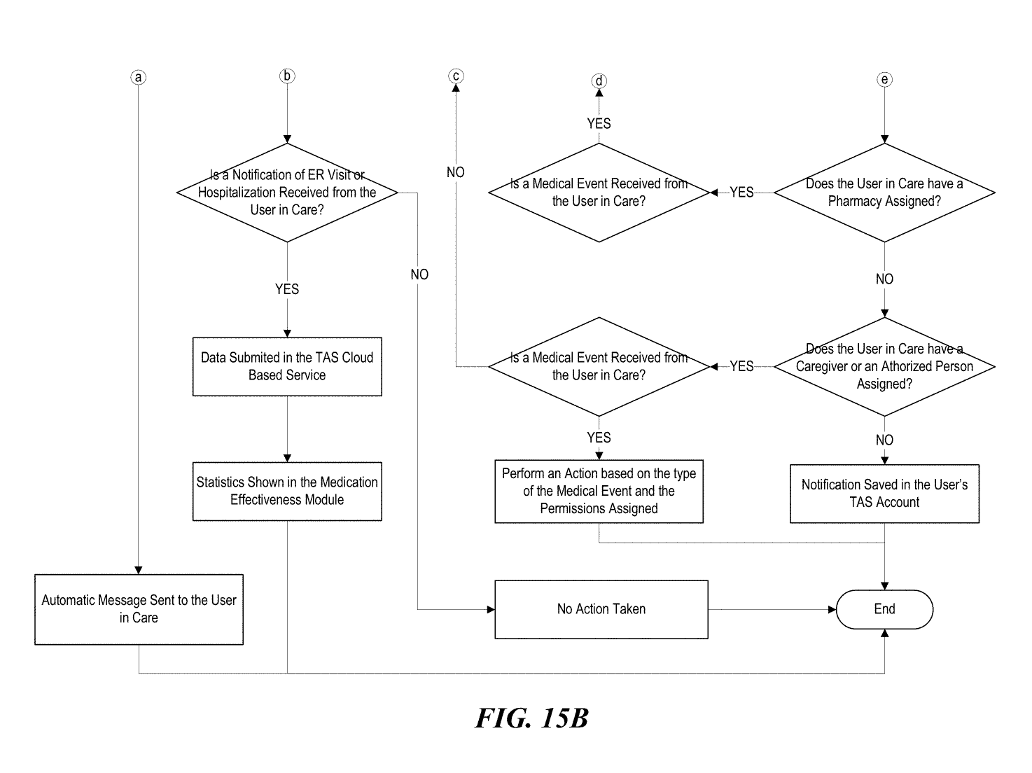

FIGS. 15A-15B are flow diagrams that illustrate a process for processing a caregiver-generated alert within a telemonitoring and analysis system.

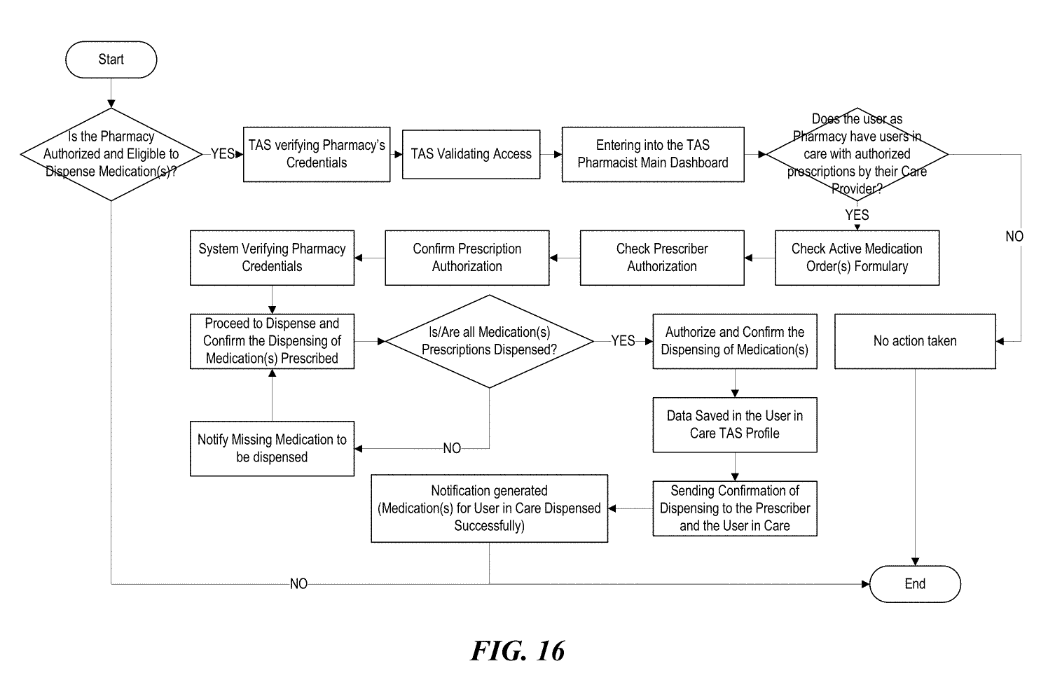

FIG. 16 is a flow diagram that illustrates a process for confirming medication orders prescribed by care providers through the telemonitoring and analysis system.

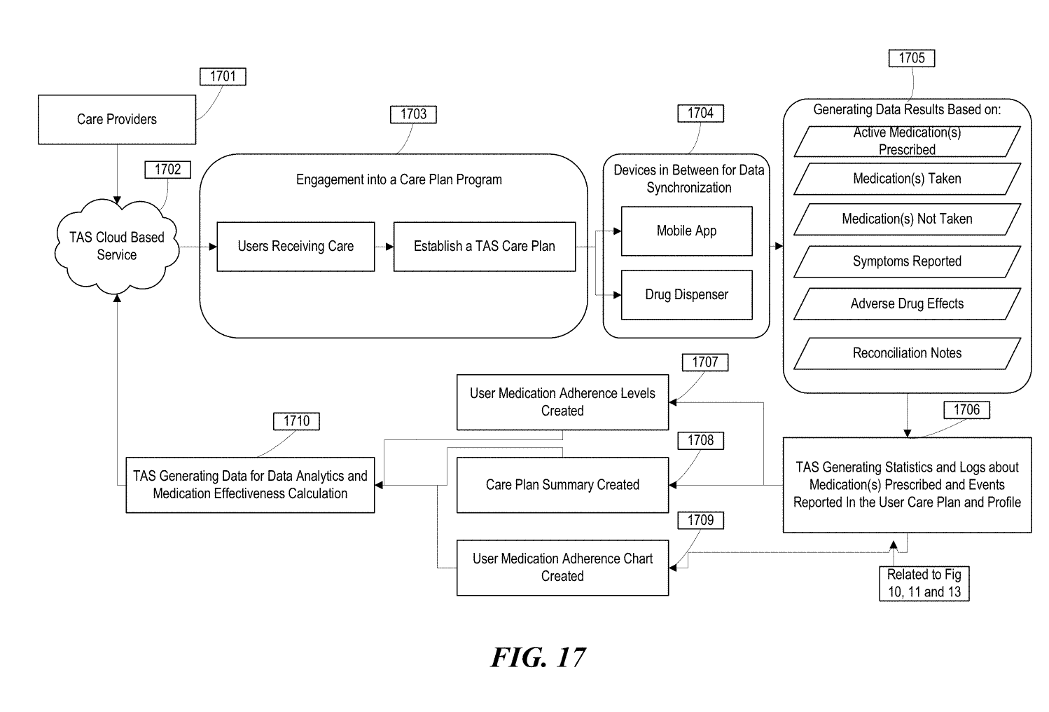

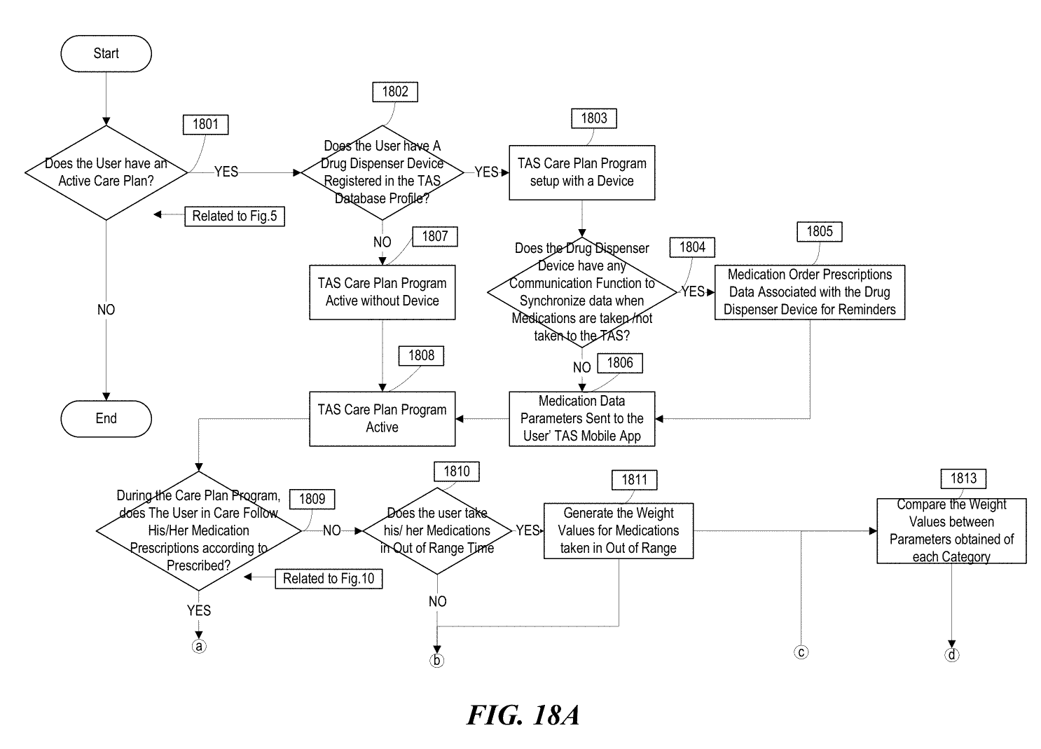

FIG. 17 is a flow diagram that illustrates a process for establishing medication adherence and compliance levels based on data obtained during execution of the care plan. In FIG. 17, data parameters are obtained during the creation of a care plan (1701, 1702, 1703). Creation of the care plan includes identifying and connecting the stakeholders and the devices that will be collecting the information related to the care plan parameters (1704). Data collection can be synchronized through the mobile application or through a medical device such as a drug dispenser device. The mobile application can remind and notify the user to take medications and to confirm whether medications have been taken. Data results can be generated based on the active medications prescribed, medications taken or not taken, symptoms reported, adverse drug effects (ADE), reconciliation notes, and other sources (1705).

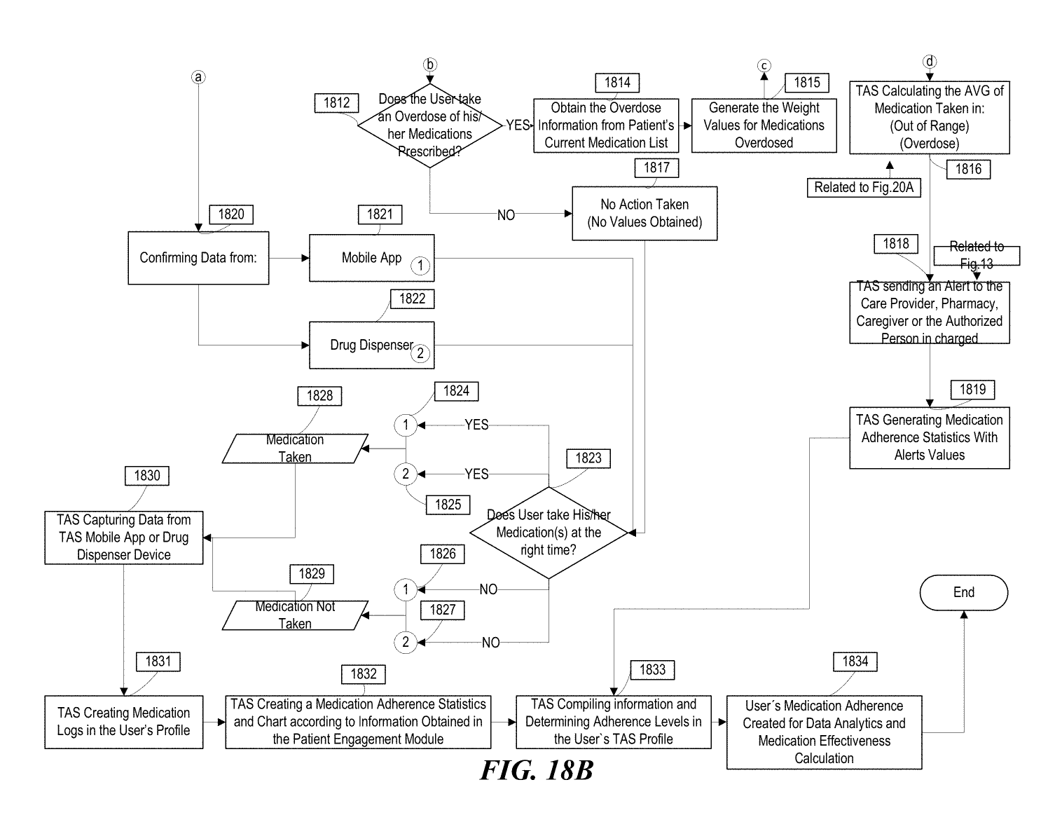

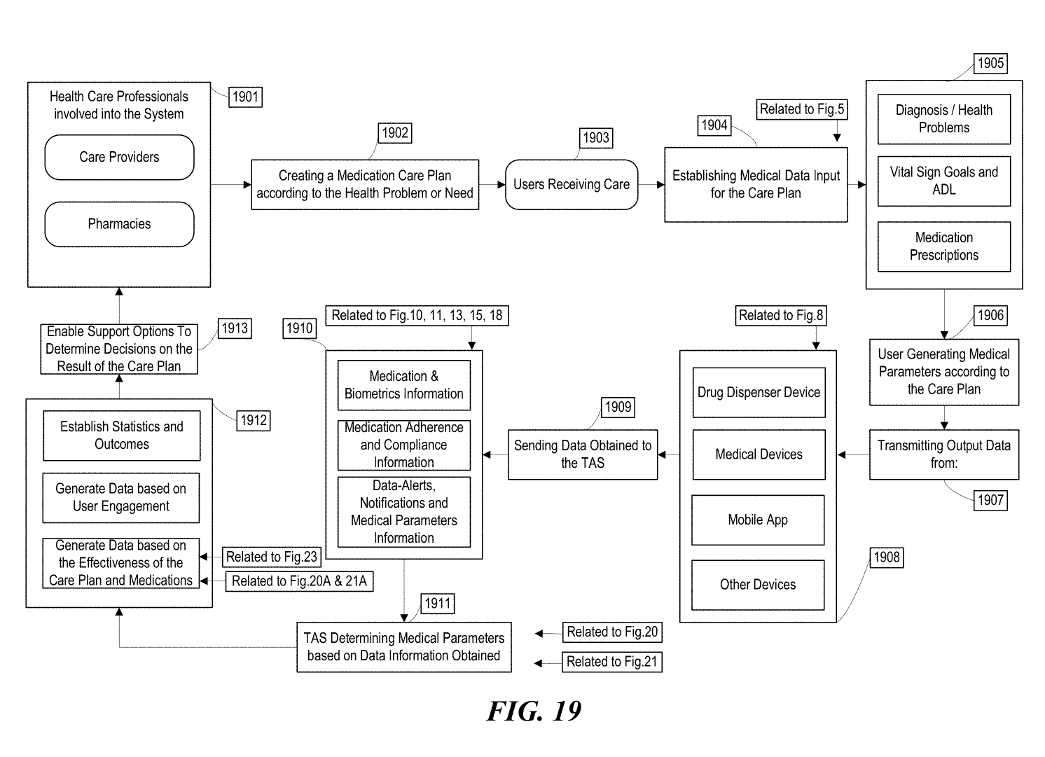

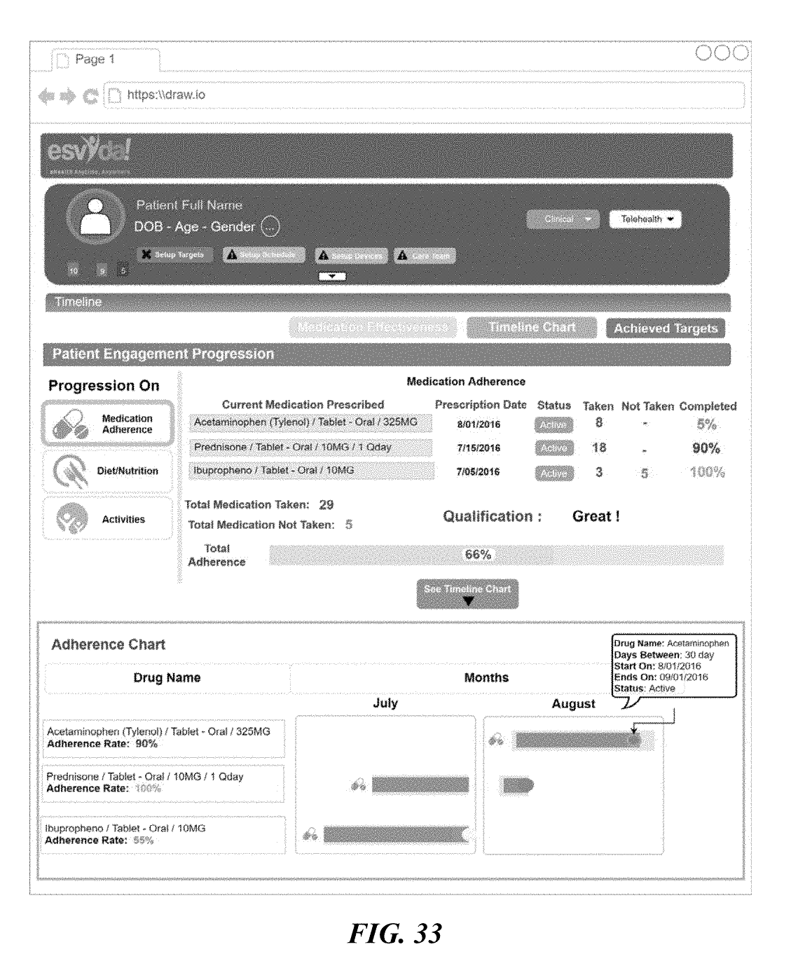

Throughout the care plan, the telemonitoring and analysis system will collect data and generate metrics associated with medication information and vital signs taken as well as the medical events (e.g., symptoms) reported by the user. Medical events can include issues that occur during the care plan related to the user's health condition. Examples of medical events include allergies, symptoms, and ADEs. Medical events can be reported by the patient, caregivers or other authorized personnel. The telemonitoring and analysis system can categorize the medical events with a weight value so that the system will calculate a total medical event score or value during the care plan. The medical event score or value can be used as a variable in determining the effectiveness of the care plan and/or medication (1706). The telemonitoring and analysis system will continuously generate statistics from the medical parameter data obtained (see, e.g., FIGS. 18A-18B) and these statistics can be displayed on a user interface (see, e.g., FIG. 33). The statistics can include the progress of the current medications prescribed and events reported in the patient profile. The telemonitoring and analysis system can create and display (or cause to be displayed) the adherence level (1707), the summary of the care plan (1708), and the medication adherence chart (1709) in the patient profile. Additionally, the telemonitoring and analysis system can use the metrics generated for the adherence levels to calculate the effectiveness of medications once medical parameters exist in the database of the patient profile (1710).

FIGS. 18A-18B are flow diagrams that illustrate a process for creating medical adherence and compliance statistics by the telemonitoring and analysis system. Beginning with FIG. 18A, the telemonitoring and analysis system determines whether the user has a care plan (1801). The telemonitoring and analysis system can determine whether the user has an assigned medical device (1802). When the user does not have an assigned device to monitor medical parameters, the telemonitoring and analysis system will automatically collect confirmed and unconfirmed medical parameter data (e.g., medications taken/not taken, vitals) from the mobile application (1807). On the other hand, if the user has an assigned device for monitoring medications or vitals, the telemonitoring and analysis system will set up the care plan with the assigned device to capture the synchronized data from the assigned device(s) and the mobile application to assimilate data as confirmed as soon as the data is generated (1803). If the device has a communication function to synchronize data (1804), then information associated with reminders (e.g., timing and dosing on prescriptions) are sent to the device (1805). If the device does not have a communication function to synchronize the data, the medical parameters are sent to the mobile application (1806). The medical parameters can be sent to the user's mobile application (1806).

Once the telemonitoring and analysis system has determined whether the user has associated medical devices, the telemonitoring and analysis system can activate the care plan (i.e., begin reminding the user to take medications/check vitals at scheduled times, gather medical parameter data, generate statistics) (1808). When the care plan is active, the telemonitoring and analysis system will collect medical parameter data according to scheduled events outlined in the care plan (e.g., medications prescribed, vitals to be taken). Either both of or one of the mobile application or assigned devices can collect information as it is generated. Collecting data from both devices and the mobile application can allow the system to compare the data to generate reliable metrics. The user can report that the user has taken a medication at the prescribed time; however, the telemonitoring and analysis system can determine that this information is false if the drug dispenser associated with the user cannot confirm that it dispensed the medication. In such situations, the telemonitoring and analysis system will take the results of the most reliable source (e.g., the drug dispenser over the user's mobile application, caregiver's mobile application over the drug dispenser data).

Each dose of medication scheduled to be taken at a certain time can be considered a “scheduled event.” When the user fails to follow the care plan by, for example, not taking a prescription on time, the telemonitoring and analysis system can receive medical parameter data stating such and will label this medical parameter data as “out of range.” The telemonitoring and analysis system can assign a weight value for this medical parameter data event based on the categorization of “out of range.” Additional weight values can be assigned for events associated with overdosing or taking the medication on time.

For example, the telemonitoring and analysis system can determine whether the user follows his/her care plan by comparing the medical parameter data for each of the scheduled care plan events with expected medical parameter data for each of the scheduled care plan events (1809). If the telemonitoring and analysis system determines that the user takes his medication in an out of range time (1810), the telemonitoring and analysis system can apply the weight values for medications taken at out of range times to the medical parameter data (1811). To calculate medication adherence levels, the telemonitoring and analysis system can compile all the medical parameter data associated with each of the scheduled events in the care plan and determine whether the medical parameter data is in range (i.e., dose taken on time and right amount), out of range (i.e., doses taken too far apart or did not take enough), or overdosed (i.e., taken two doses without enough time in between or took too much) by comparing expected times/amounts in the scheduled events outlined in the care plan (1811, 1812, 1813, 1814, 1815). The telemonitoring and analysis system can compare the weighted values between the medical parameter data obtained in each category (e.g., out of range, in range) and calculate an average (1816). The average can be used as a factor or variable in determining an adherence level.

If the medical parameter data indicates that the user did as he was supposed to (i.e., took medication within a range and the user did not overdose) (1817), the telemonitoring and analysis system can document the event but create a value of zero for the event and can proceed with calculation of the adherence levels. After the medical parameter data is obtained from the mobile application and/or medical devices, the telemonitoring and analysis system creates corresponding logs of medications taken at the correct time, not in range and overdosed in the patient profile to create the total adherence levels and statistics (1820, 1821, 1822, 1823, 1824, 1825, 1826, 1827, 1828, 1829, 1830, 1831, 1832). During the care plan treatment, when medications/vitals are taken during an out of range time or when the medications/vitals are not taken, the system can generate alerts within the patient profile so that caregivers and others are aware of the potential issues (1818, 1819). The alerts can be shown in an adherence progress chart with an indication of the time and date in which the alert occurred. When the medical parameter data has been collected and metrics have been generated for all the scheduled events during the care plan, the telemonitoring and analysis system can compile all the medical parameter data to create an adherence level of the patient (1833). The adherence level can be used as a category or variable in calculating the effectiveness of the care plan (1834).

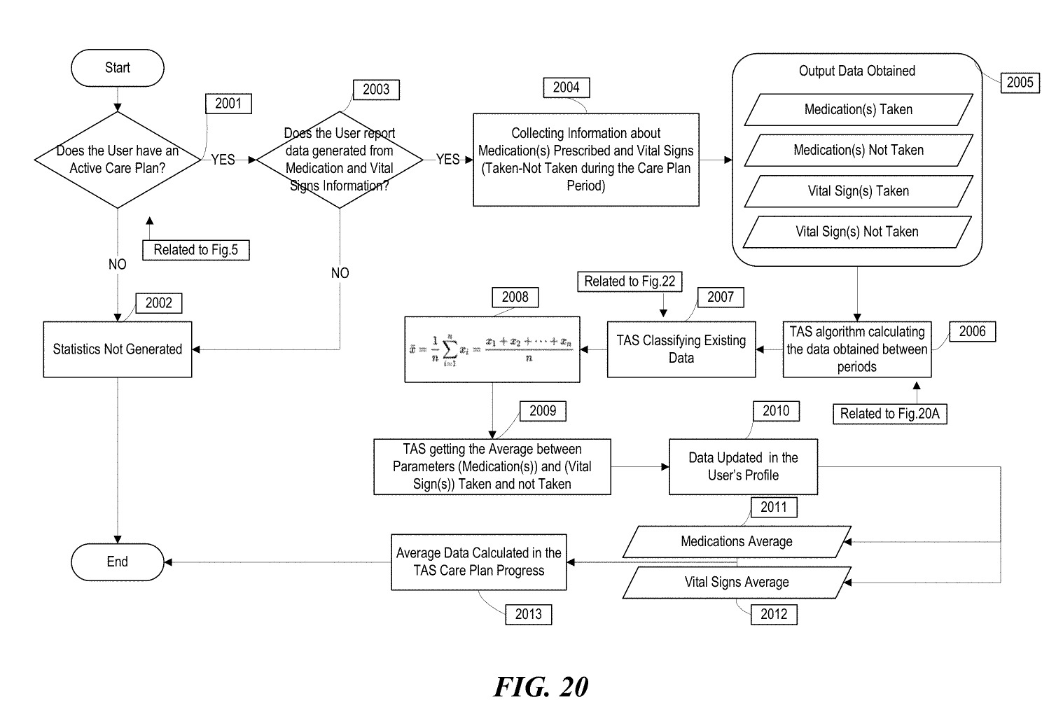

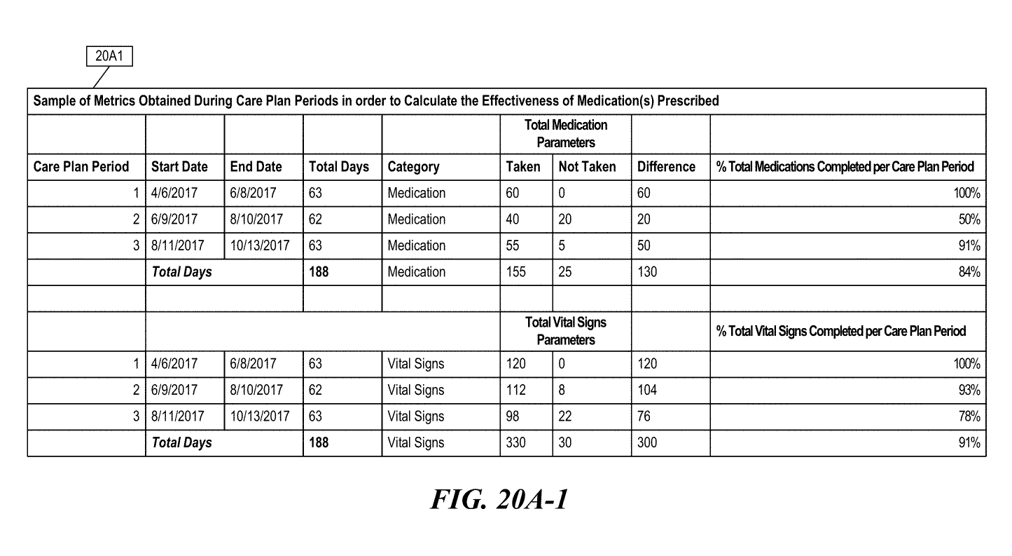

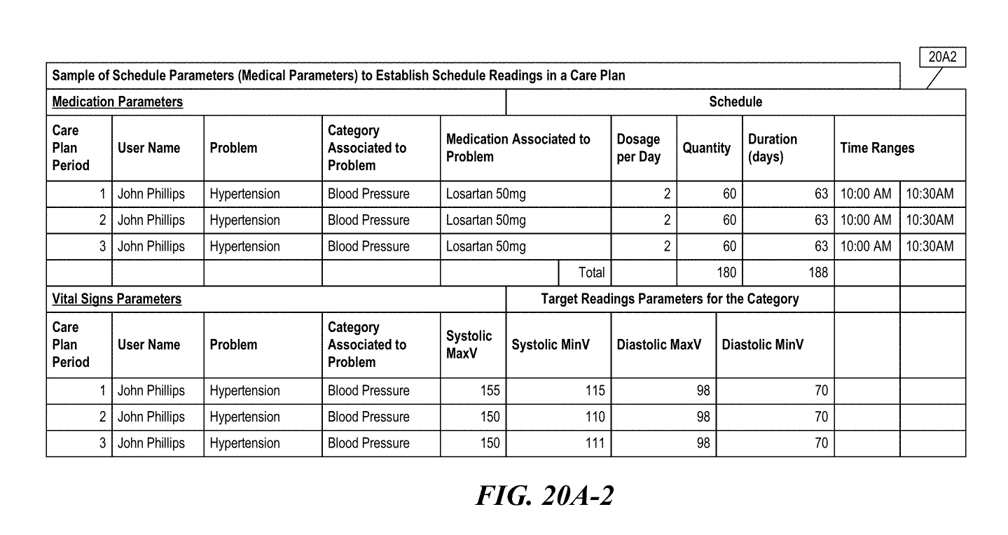

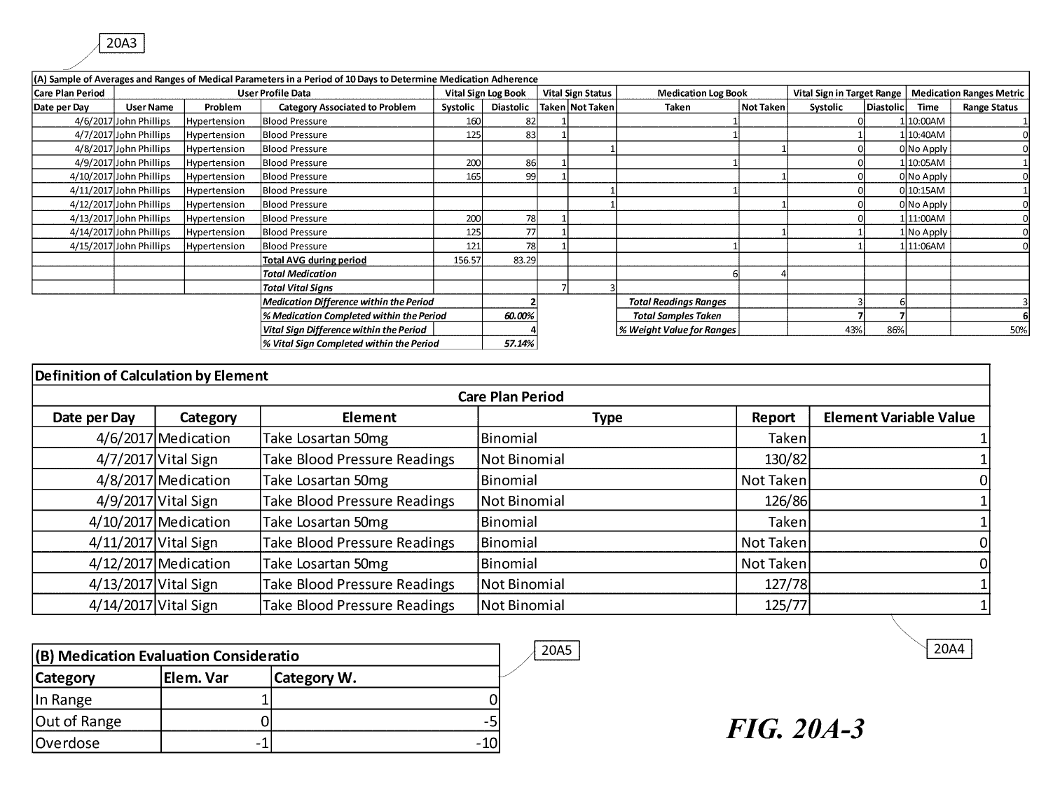

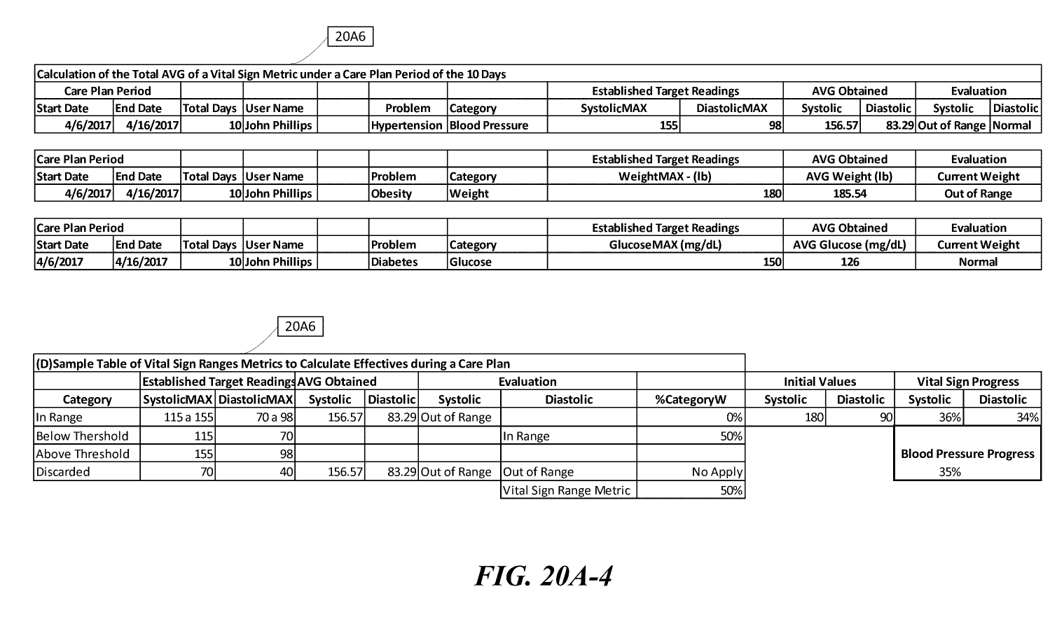

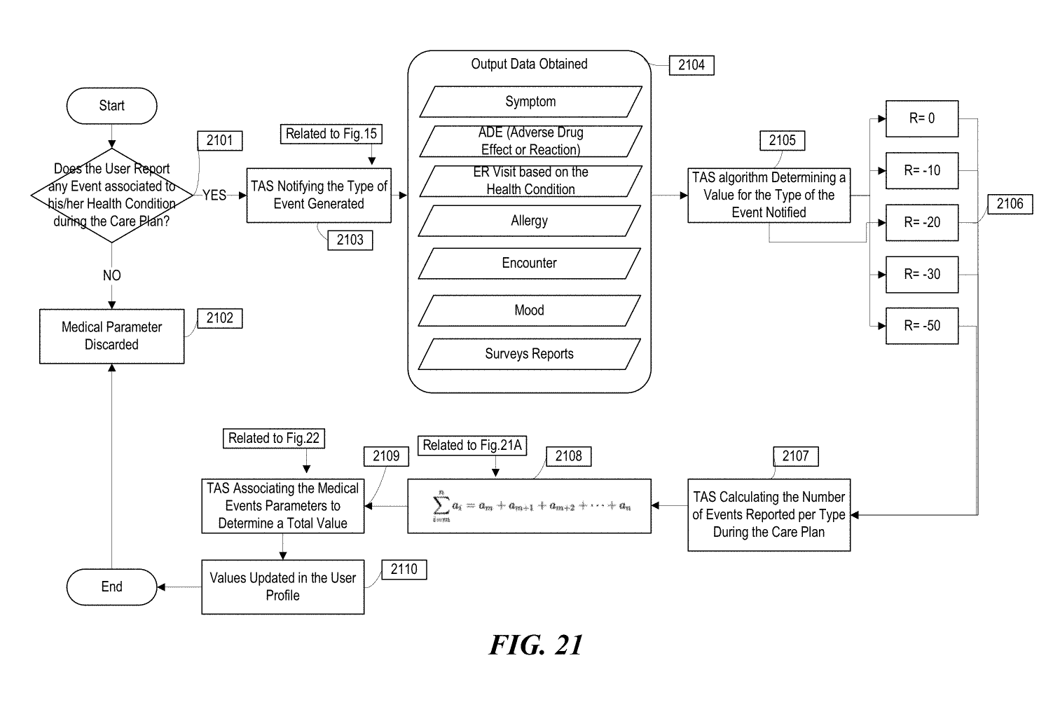

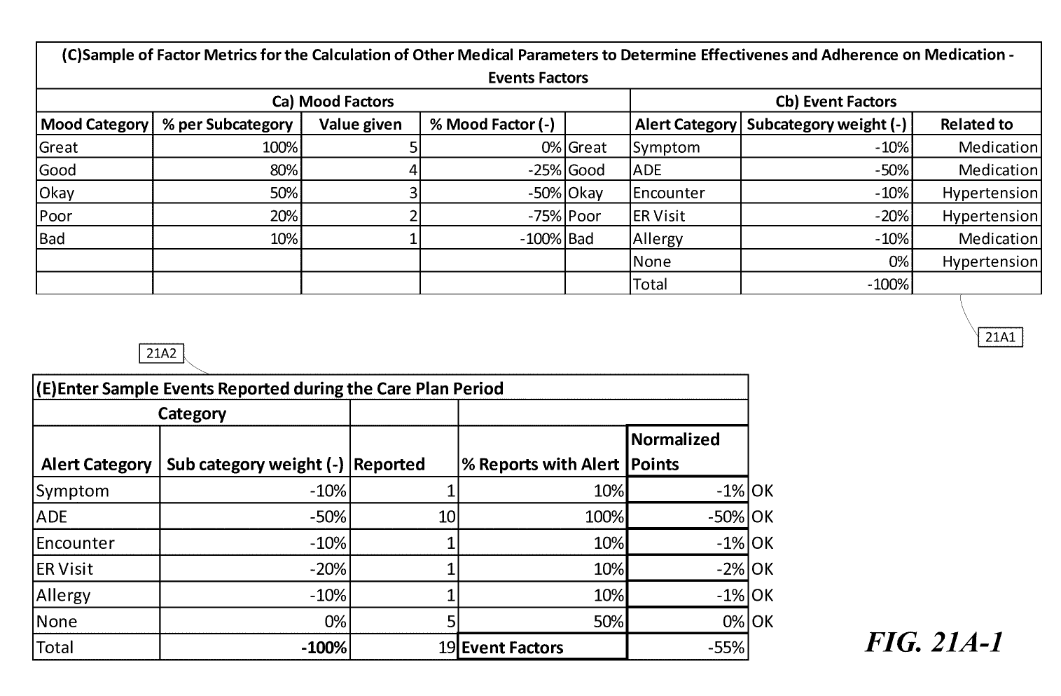

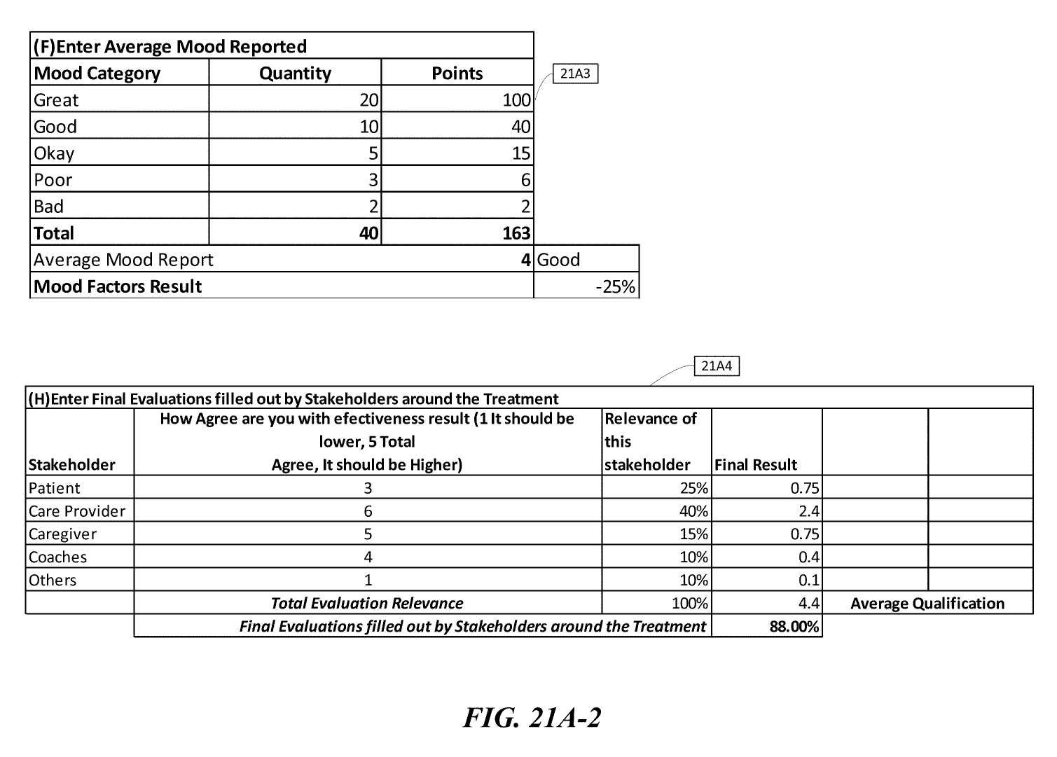

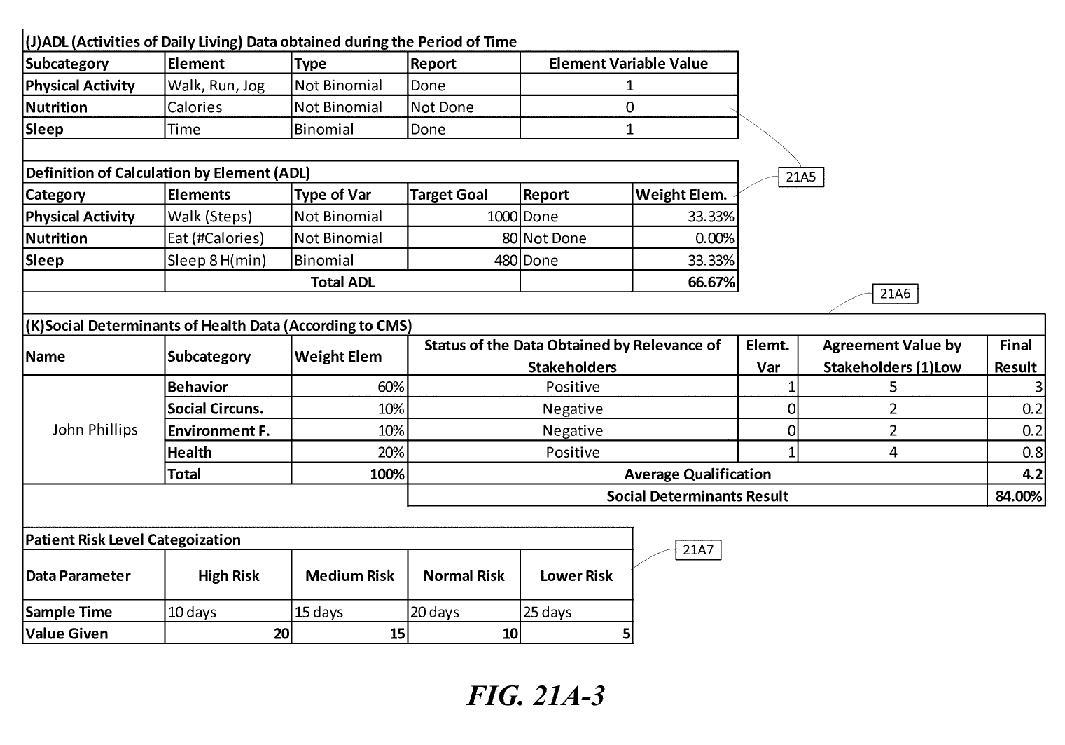

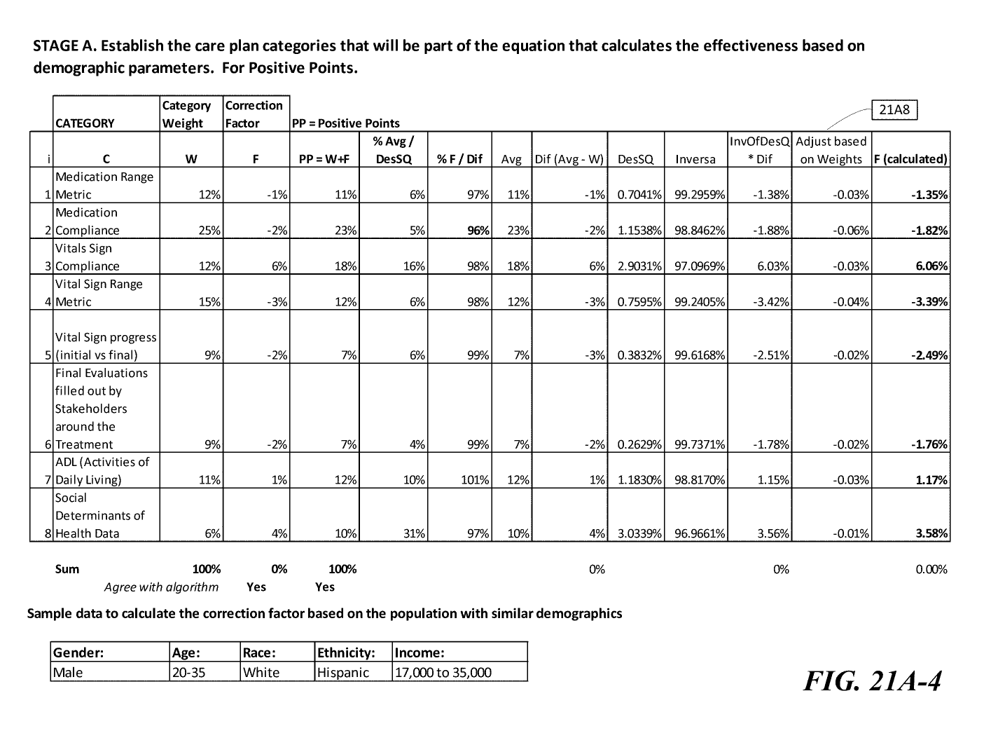

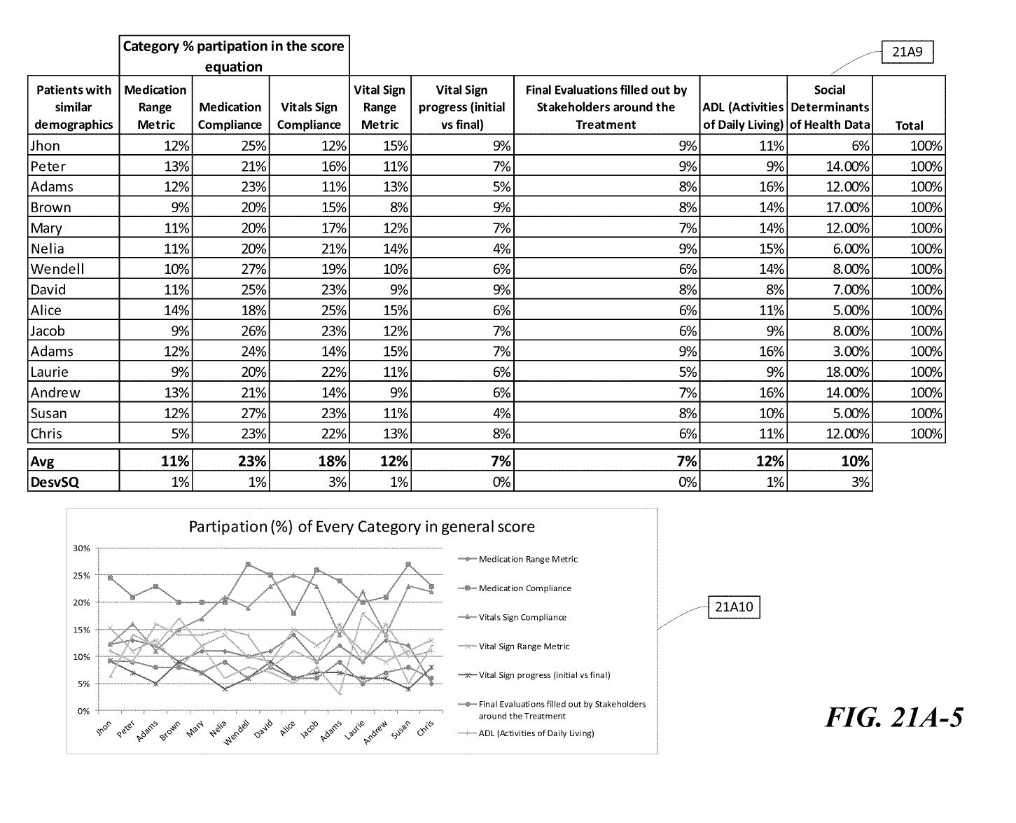

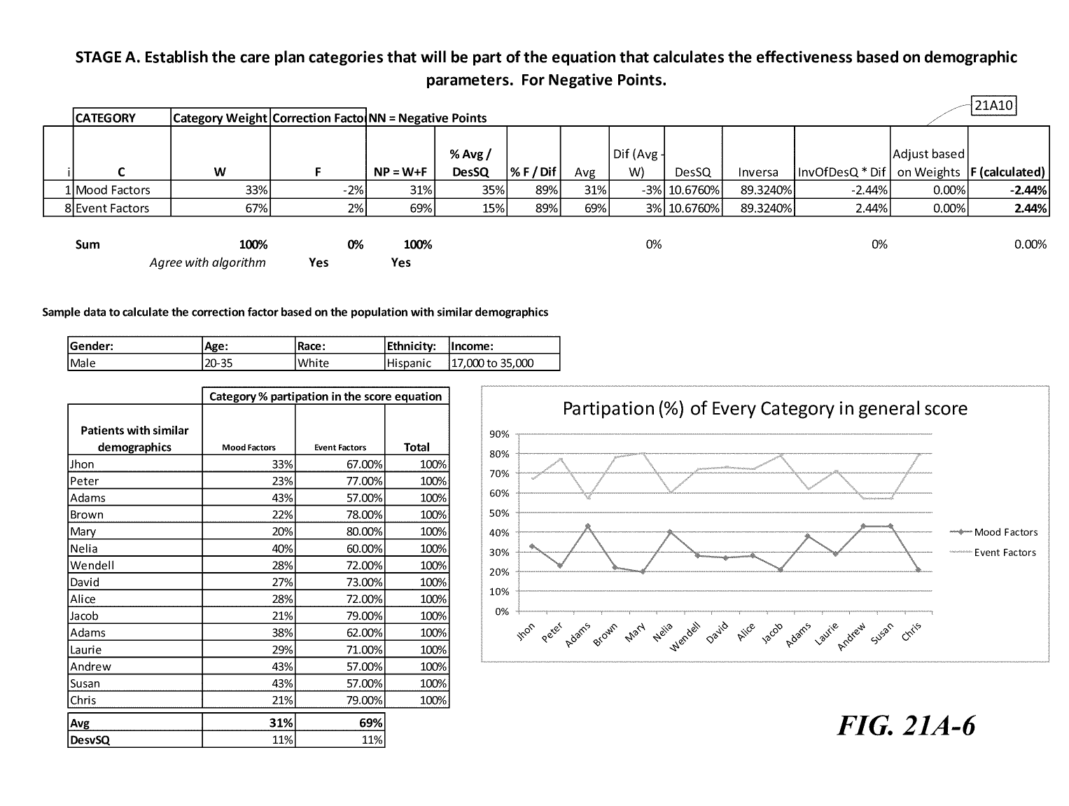

FIG. 19 is a flow diagram that illustrates a process for determining effectiveness of a care plan using a telemonitoring and analysis system. The process illustrated in FIG. 19 summarizes the processes illustrated in FIGS. 20A-20D and FIG. 21, including the data obtained during a care plan used to determine the effectiveness of the care plan. The effectiveness can be based on categories such as medical parameters, medical events or alerts, moods, ADL, comments from others, and surveys. During the enrollment of a patient into the telemonitoring and analysis system, care providers can select which patients need a care plan. After the patient evaluation and diagnosis are completed, either during an in-person consultation or a virtual visit, the telemonitoring and analysis system will store the input data parameters such as current patient diagnosis/health problem to treat, scheduled readings and goals for medications/vital signs, and plan in a database and use the input data parameters as a basis for the calculation of results given during the treatment (1901, 1902, 1903, 1904, 1905).

As mentioned before, during the care plan, medical parameter data and medical events will be generated depending on the patient, the diagnosis and the care providers or other stakeholders (1906, 1907). The medical parameter data, medical events and other data can be transmitted via a medical device assigned to the patient such as a drug dispenser device, a WIFI direct or Bluetooth device, the telemonitoring and analysis system mobile application or any other device that sends information electronically (1908, 1909). The telemonitoring and analysis system will categorize the output data generated by the patient during the care plan into his/her profile (1910).

The collected data can be used to calculate an effectiveness of the care plan. The collected data can include medication and biometrics information, medication adherence and compliance information, data alerts, notification, and medical parameters information. Such data can be stored in the patient's profile and can be categorized in parameters or factors, each with a unique value for the algorithm that calculates effectiveness (1909, 1910, 1911). Finally, the process concludes with the generation of statistics, outcomes and engagement levels along with the effectiveness progress to determine the total progress of a patient following a care plan (1912, 1913).

FIG. 20 is a flow diagram that illustrates a process for determining the variables that will be used in calculating the progress of medical parameters using a telemonitoring and analysis system. The telemonitoring and analysis system first determines whether the user has an active care plan (2001). If not, no statistics are generated (2002). On the other hand, if the patient has an active care plan, the telemonitoring and analysis system can receive medical parameter data (e.g., medication and vital sign information) when such data is generated (2002, 2003). When such data is generated, continuous statistics on the categories associated with the patient in the care plan are determined based on the data.

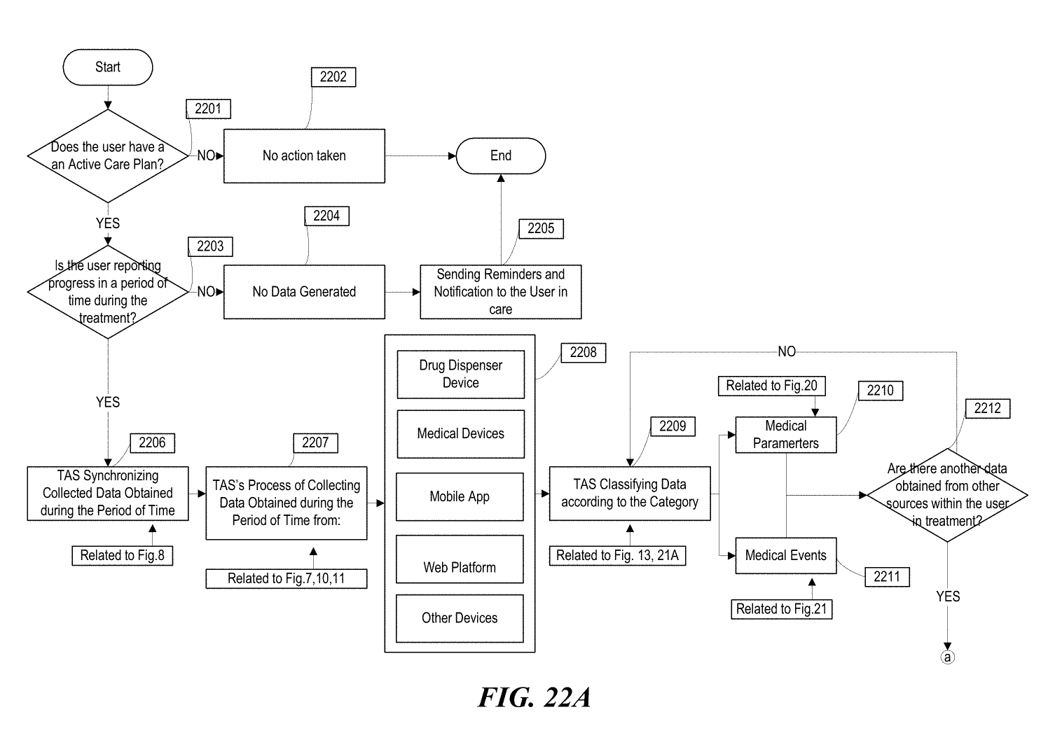

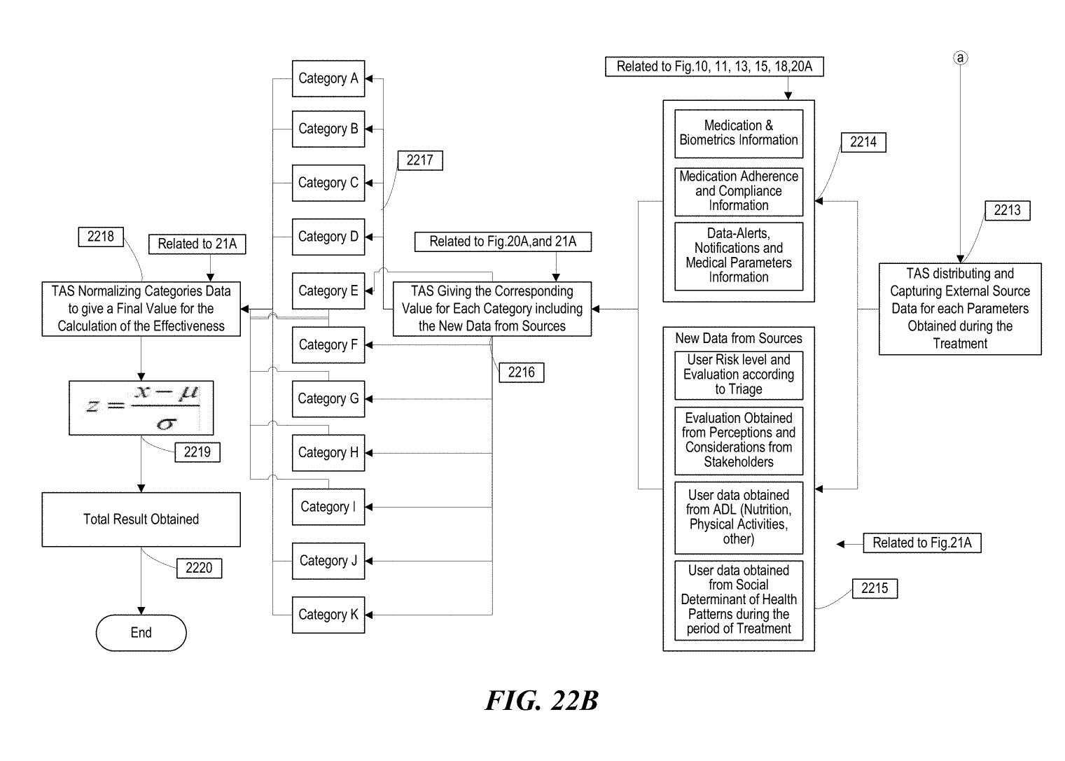

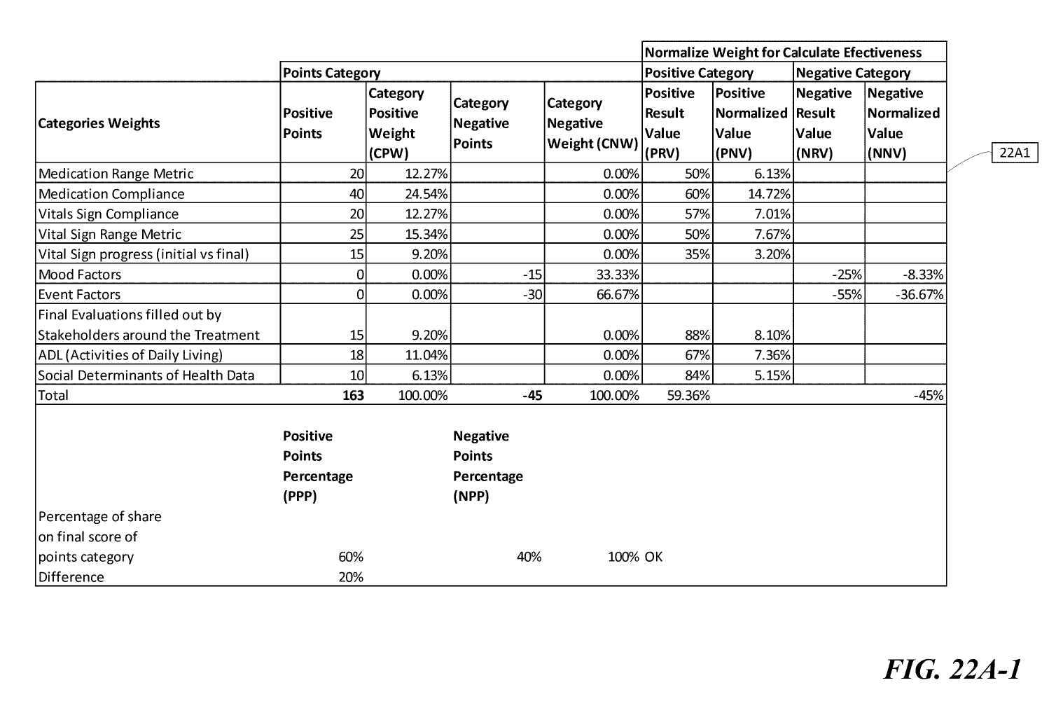

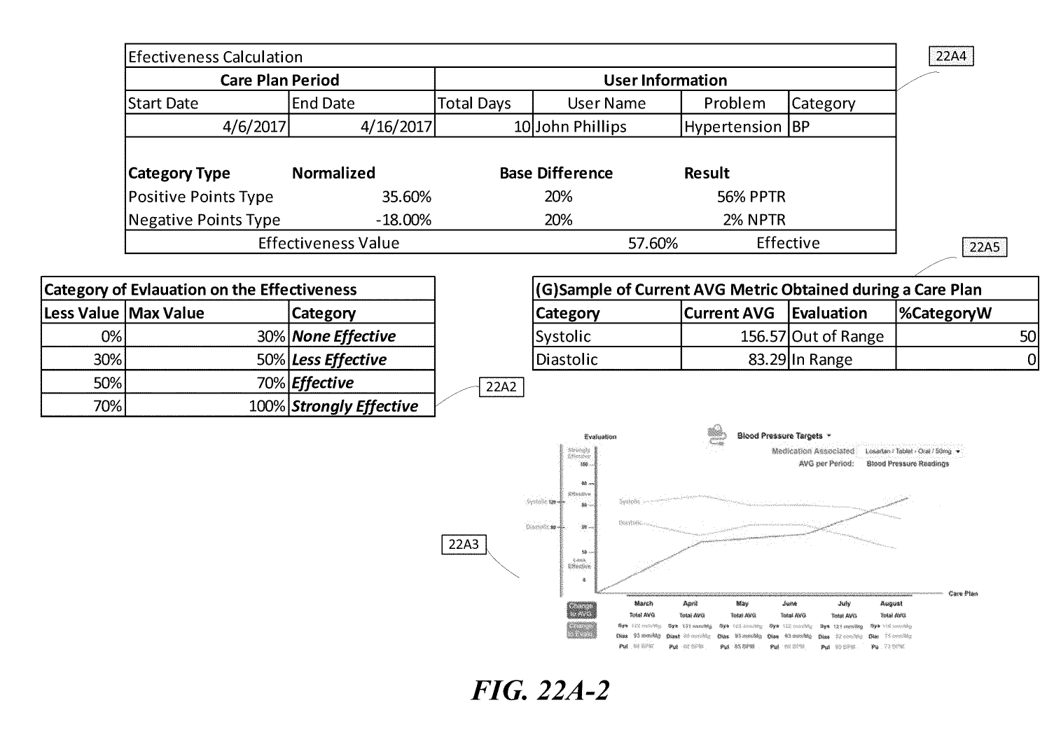

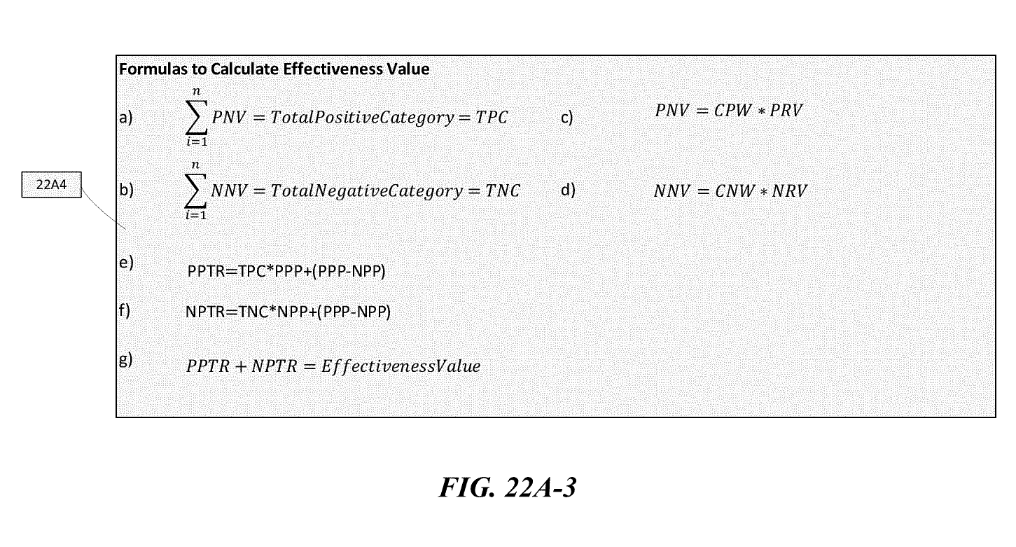

As mentioned, medical parameters are parameters associated with previously scheduled vital signs or medication data (e.g., medication taken, medication not taken, vital signs taken, vital signs not taken) and medical parameter data associated with the medical parameters can be obtained during the execution of a care plan (2004, 2005). The medical parameter data received by the telemonitoring and analysis system can be categorized as unique values (e.g., adherence levels) and used in the total calculation of medication effectiveness. Other medical parameter data such as scheduled ranges and alerts (discussed in FIG. 18A-18B) can be included. In some embodiments, the classification of existing data can relate medical events to medical parameters. This classification is further explained in FIG. 22A-22B. The data values can be used to create progress values of the patient during periods of time.

The telemonitoring and analysis system can calculate the data obtained between periods (2006), classify the existing data (e.g., in range, out of range) to determine values (2007), and average the results of the medications adherence levels and the vital signs adherence levels (2008, 2009) to determine one or more adherence levels. The patient profile will be updated with the average medication adherence levels and the average vital signs adherence levels during the care plan (2010, 2012, 2012, 2013, 2014). In some embodiments, the care plan can have several associated periods of time. Each period can include many different scheduled events. In some embodiments, the progress values of the patient can be created during periods of time.

FIGS. 20A-1, 20A-2, 20A-3, 20-A-4 illustrate the calculation and show examples of categories that can serve as variables in calculating effectiveness of the care plan (2006, 2007, 2008, 2009).

FIG. 20A-1 illustrates a sample of medical parameter data obtained during a care plan period to calculate the effectiveness of a medications prescribed (20A1).

FIG. 20A-2 illustrates a sample schedule of medical parameters that can be used to establish scheduled readings in a care plan (20A2). The medical parameter data received from the mobile application and various medical devices can be compared to expected medical parameter data.

FIG. 20A-3 includes a sample of averages and ranges of medical parameters during a period of time (20A3). Various statistics can be calculated including the percentage of medications completed during the period as well as the percentage of vital signs completed within the period. Each element can be categorized as binomial or not binomial and the report varies depending on the type of element (e.g., taken/not taken, a number such as a blood pressure). Further, each element can be given a value. Moving to 20A5, the medical parameter data can be classified into scoring categories (e.g., in range, out of range, overdose) based on the comparison. The system can then assign a weighted value to the medical parameter data for each of the scheduled care plan events. The weighted value can be based on the scoring class. For example, an event that is determined to be an overdose scores a negative 10 points. The telemonitoring and analysis system can average the weighted values of the medical parameter data for each of the medical parameters to determine an adherence level.

FIG. 20A-4 illustrates a sample calculation of a total average of a vital sign metric over a period of time (20A6). In this example, the average systolic reading is out of range, the diastolic reading is normal, the average weight is out of range, and the average glucose is normal. This data is used in 20A7, which provides a sample table of vital sign range metrics. Category weights are applied to obtain adherence levels (e.g., 50%) and progress reports (e.g., 35% better than the initial values).

FIG. 21 is a flow diagram that illustrates a process for determining the variables that will be used in calculating the progress of the user based on medical events using a telemonitoring and analysis system. The process begins when the telemonitoring and analysis system determines whether the patient reports any events associated with his health condition during the care plan (2101). If not, the medical event parameter is discarded (2102). When medications are prescribed, several factors can affect the progress of the effectiveness. For example, a patient can report adverse reactions towards medications being taking to treat the condition. The telemonitoring and analysis system can be notified of the event (2103) and can categorize the medical event (e.g., alert, mood, survey) (2104) and such events can be subcategorized (e.g., alerts can be subcategorized to symptoms, ADE, encounter, ER visit, allergy). The patient can document any medical event associated with the condition. In some embodiments, others can report the medical events (e.g., emergency room visit can be reported by third party). Events related to the condition and medication prescribed can create the variables used to calculate the medication effectiveness of the patient during the care plan. In some embodiments, additional “events” are received from external sources such as surveys. The survey can be an evaluation of care plan progress of the patient by a stakeholder (e.g., caregiver). Each medical event can be scored slightly differently as each medical event will have different subcategories.

The telemonitoring and analysis algorithm can calculate medical event scores for each category of medical event (e.g., mood, survey, alert). The telemonitoring and analysis system can further subcategorize the categories of medical events. The medical event scores can be unique values used as additional parameters or factors in calculating the effectiveness of the medications and the care plan (2105, 2106). In addition, as discussed above and shown in FIGS. 20A-1 to 20A-4, the telemonitoring and analysis system can give weighted values for each subcategory for medical events reported by a patient. A final medical event score can include a score reflecting a number of events reported per type (e.g., two trips to the ER), corresponding alerts and can take into consideration the level of patient risk to modify the algorithm behavior (e.g., the algorithm will calculate the progress score more frequently for patients at high risk than for the patients at a lower risk (2107, 2108). The telemonitoring and analysis system will sum each type of medical event reported with the total values per type and will determine the final results of the medical event scores in order to create the parameter needed to be included into the algorithm that will calculate the effectiveness of the care plan (2109). In some embodiments, the effectiveness of the care plan is reflective of the effectiveness of the medications taken during the care plan and in other embodiments, the effectiveness is the effectiveness of the care plan itself. Finally, the progress levels of the patient for each category of evaluation (medication adherence, medication compliance) can be updated in the patient profile (2110).

FIGS. 21A-1, 21A-2, and 21A-3 illustrate an example of data and a calculation of metrics based on medical events and external source parameters, consistent with various embodiments.

FIG. 21A-1 illustrates a sample of factor metrics for the calculation of certain medical event parameters (e.g., mood category, alert category) (21A1). Each category (e.g., mood category, alert category) can have subcategories and each subcategory can be weighted. Additionally, the categories can be associated with medications or conditions.

FIG. 21A-2 illustrates an example of a medical event of mood reports and the associated scoring (21A3) and an example of a medical event of survey reports and the associated scoring (21A4).

FIG. 21A-3 illustrates an example of a medical event of ADL and the associated scoring (21A5), social determinants of health data and the associated scoring (21A6), and categorizations for patient risk levels (21A7). If the patient is at a higher risk, the sample time for obtaining and analyzing data is shorter and a higher value is given.

FIG. 21A-4 illustrates an example of applying a correction factor to the variables used to compute the effectiveness (e.g., adherence levels, medical event scores). In this example, the correction factors are calculated for the factors having a positive weight. The correction factor can be based on external sources, expert considerations, analysis of average, variance (sum of the square) and covariance of weights assigned to other individuals with similar demographic characteristics (e.g., age, gender, ethnic origin, race, economic status). Calculating the correction factor can include average, sums of the square of deviation of weights assigned to other individuals with similar demographics characteristics and data normalization of the correction factors, so that the summation of the weights modified by the correction factor will be equal to one hundred.

FIG. 21A-5 illustrates an example of participants and the weights applied to the variables used to calculate effectiveness (e.g., vital signs compliance, vital signs range metric, vital sign progress, final evaluations, ADL, social determination of health data) (21A9). The telemonitoring and analysis system can generate a participation percent of every category in the general score (21A10).

FIG. 21A-6 illustrates an example of applying a correction factor to the variables used to compute the effectiveness (e.g., mood factors, event factors). In this example, the correction factors are calculated for the factors having a negative weight. The correction factor can be based on external sources, expert considerations, analysis of variance and covariance of weights assigned to other individuals with similar demographic characteristics (e.g., age, gender, ethnic origin, race, economic status).

FIGS. 22A-22B are flow diagrams that illustrate a process for classifying medical parameters and medical events to calculate effectiveness of a care plan (including medication effectiveness) by a telemonitoring and analysis system. When a user does not have an active care plan, no action is taken (2201, 2202). When a user engaged in an active care plan reports progress including alerts notifications, these reports are synchronized through any supported or associated device and are collected by the telemonitoring and analysis system (2201, 2203, 2204, 2205, 2206, 2207, 2208). The telemonitoring and analysis system will classify the data per category established by the system as unique parameter based on initial experts and the medical parameters and medical events collected during the creation of the care plan. In addition, the telemonitoring and analysis system will take into account external information gathered from external sources such as patient risk level, evaluation from perceptions and considerations from stakeholders regarding to patient health, patient ADL, prescriptions and social determinants of health. Social determinants of health refer to the final perception relating to the patient's taking of the medication as reported by the stakeholder responsible for the care plan. According to CMS studies, the initial expert's values for each determinant such as (Behavior, Social Circumstances, Environmental Factors, and Health) are included in the algorithm that determines the corresponding weight value for this category (2209, 2210, 2211, 2212, 2213, 2214, 2215). The values for each category obtained during the care plan will be given a weight value, a given value and total result depending of the type of category that requires calculation of different variables obtained. As discussed with regard to the previous figures, the total calculation of each of the category scores will be normalized to determine a final outcome on the medication effectiveness. This data result now will be calculated in the algorithm established for the determining either the effectiveness of the medications prescribed in periods of time or the effectiveness of medications according to medical events reported (2116, 2117, 2118, 2119, 2120).

FIGS. 22A-1, 22A-2, and 22A-3 illustrate an example of data and a total calculation between medical parameters, medical events, and external sources to determine effective of the care plan, consistent with various embodiments. FIG. 22A-1 illustrates categories, their associated points and normalized weights (22A1). FIG. 22A-2 illustrates an effectiveness calculation for a patient during a care plan (22A4), categorization of evaluation on the effectiveness (22A2), sample of average metrics (22A5) and a chart (22A3). FIG. 22A-3 illustrates the formulas that can be used to calculate an effectiveness value.

FIGS. 23A-23B are flow diagrams that illustrate a process for determining an effectiveness of a care plan by a telemonitoring and analysis system. The final calculation of the medication effectiveness of a care plan period can be based on different data parameters and categories given by initial experts, considerations and can be dynamically adjusted with final evaluations filled out by patients, care providers, caregivers, coaches, and other participants associated with the patient treatment. The telemonitoring and analysis system collects holistic elements to forecast medication effectiveness based on the comparison of adherence patterns reported from other individuals against patient treated. Those include medical parameters, medical events and external sources parameters.

The telemonitoring and analysis system starts by collecting medical parameters such as the total vital signs and medication taken and not taken during the care plan (2301, 2302). If the data-alerts generated during the period are related to either medications or vital signs, the telemonitoring and analysis system will classify the data and will determine the weight category values of each parameter reported (2303, 2304, 2305, 2306, 2307, 2308). If the care plan data does not require modifications, the final outcome will not be altered and will sum the current values and parameters obtained (2309, 2310, 2311). The telemonitoring and analysis system can show the current progress values over the period of time (2312). Once unique values for the categories are normalized, the telemonitoring and analysis system generates the final results for the calculation of the effectiveness of medication based on the care plan (2313). The telemonitoring and analysis system can perform the calculation as described in FIG. 22A-22B to give the final result on the effectiveness and will be updated in the interface of the patient in care. Care providers can determine the modifications to be made to the treatment or they can decide to change the care plan by creating a new initial diagnosis based on the medication history and health progress status (2314, 2315, 2316, 2317).

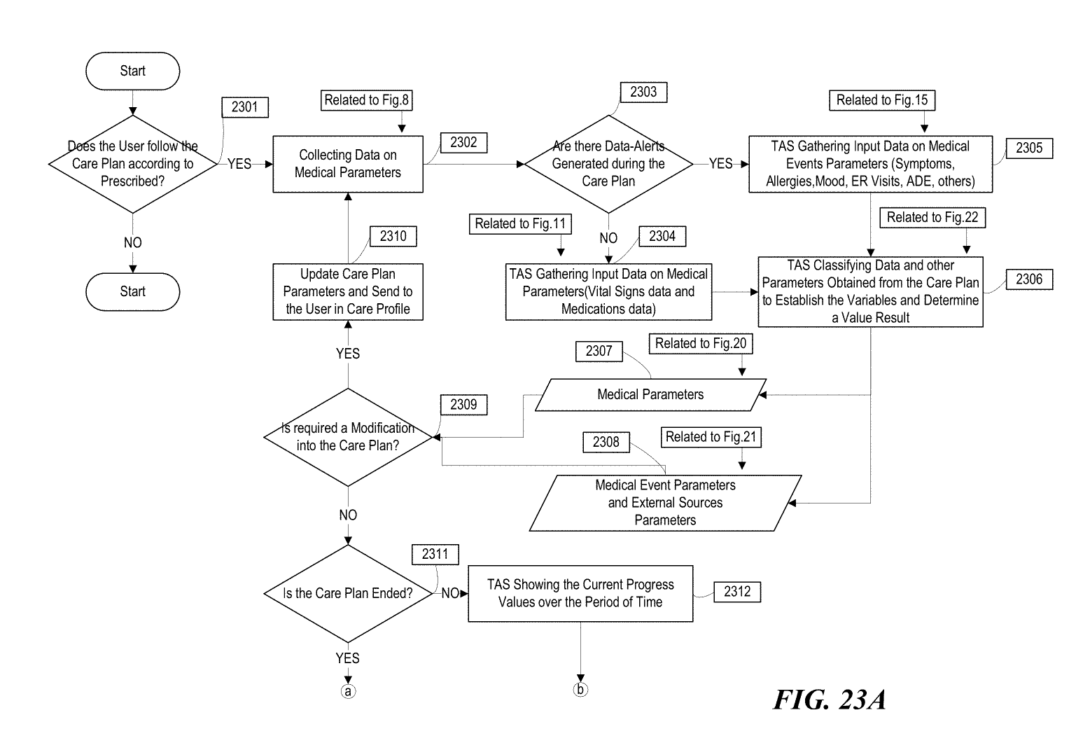

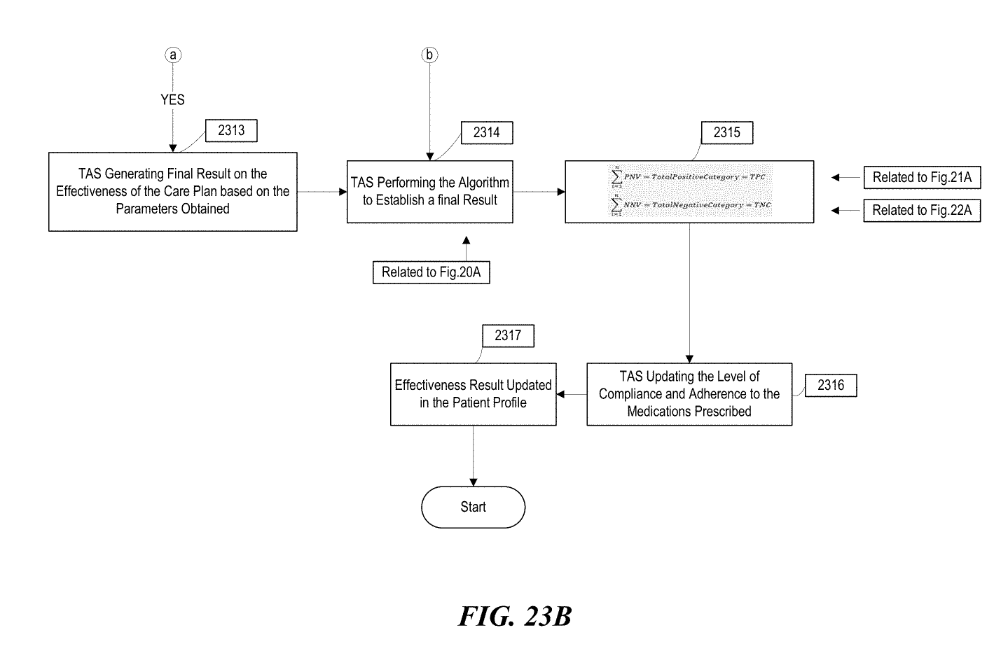

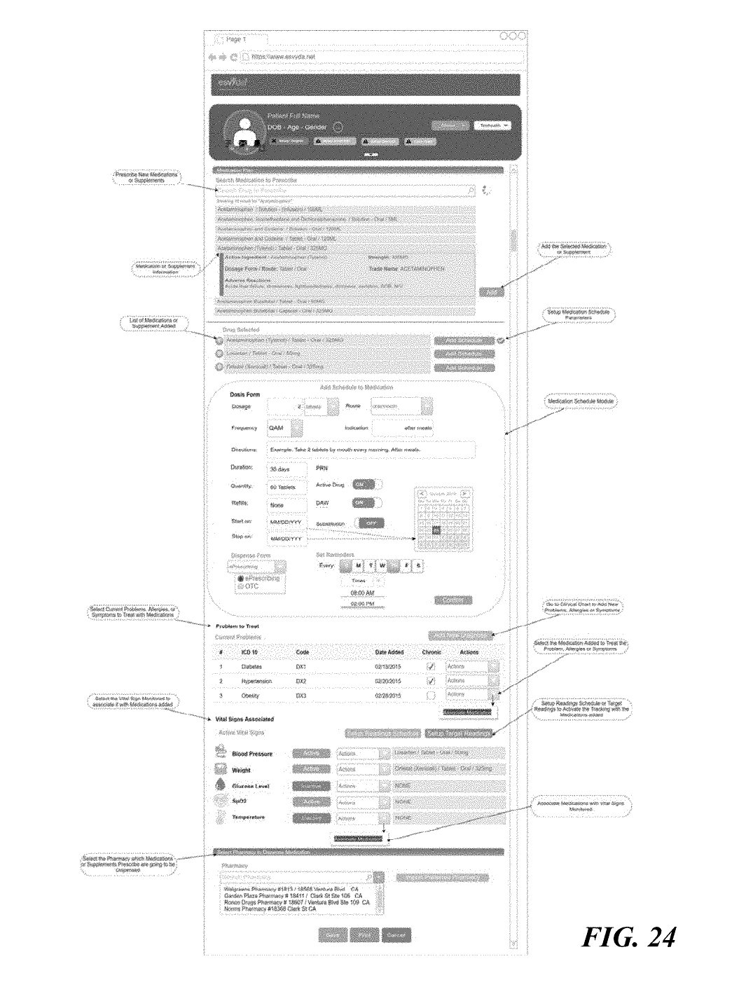

FIG. 24 illustrates an example of an interface that can be used for medication management of users in a telemonitoring and analysis system, consistent with various embodiments.

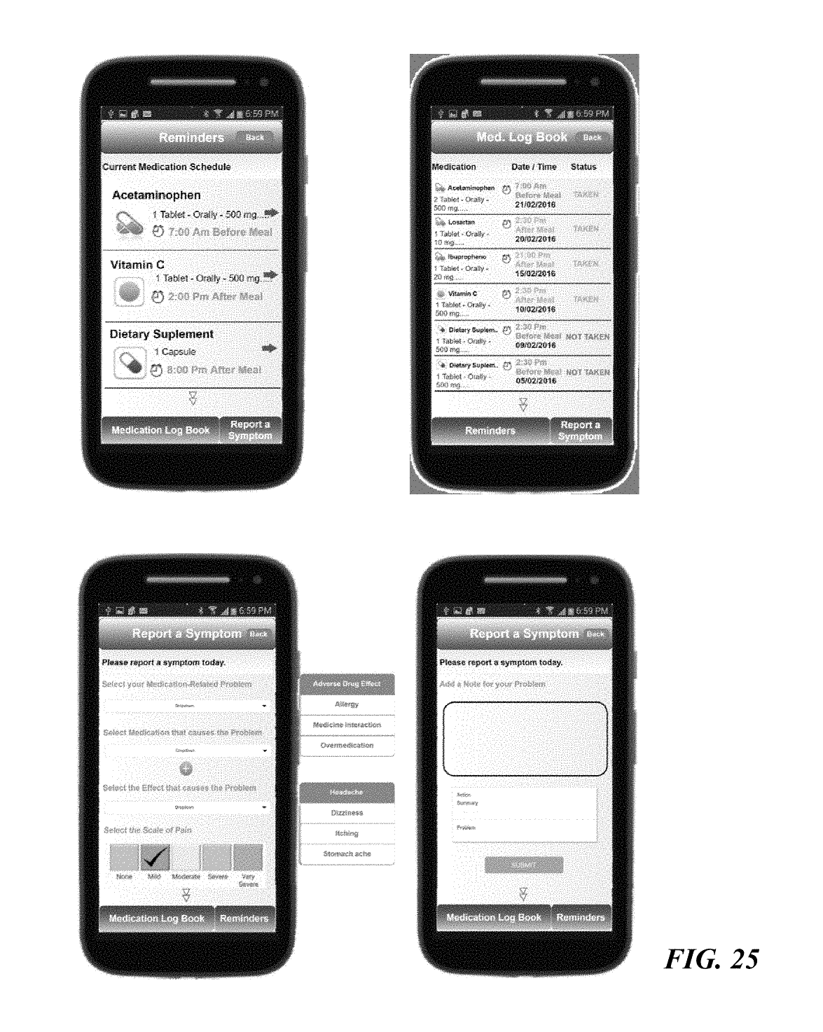

FIG. 25 Illustrates an example of medication care plan module interfaces of the telemonitoring and analysis system, consistent with various embodiments.

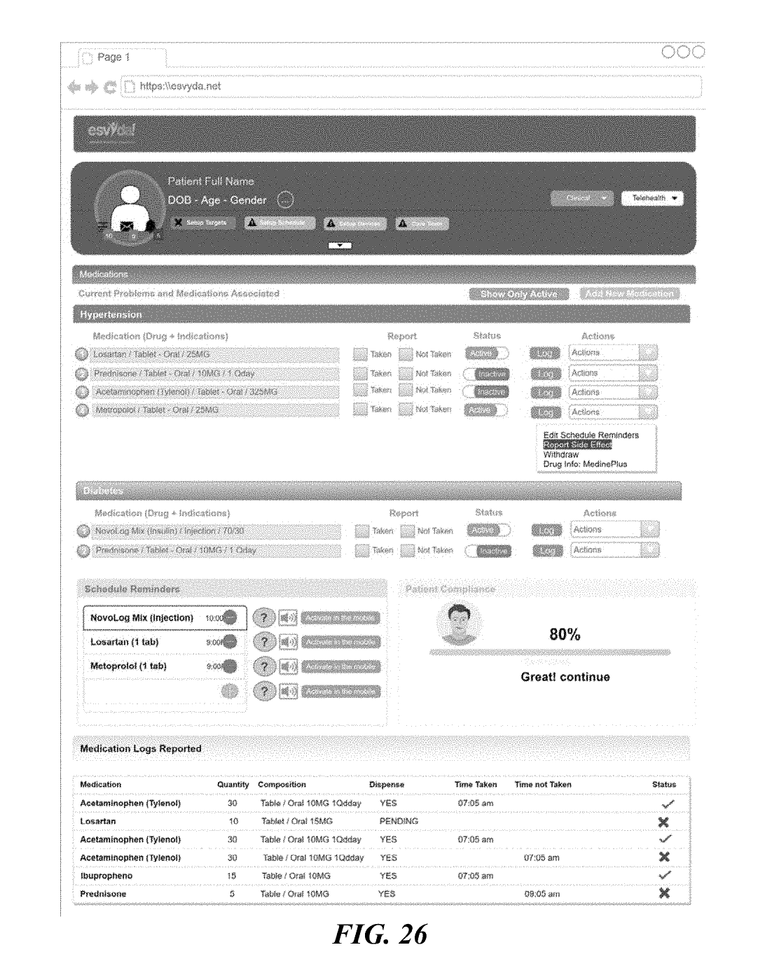

FIG. 26 illustrates an example of a medication module interface of active and inactive medications prescribed to treat health conditions and a medication compliance score of a user, consistent with various embodiments.

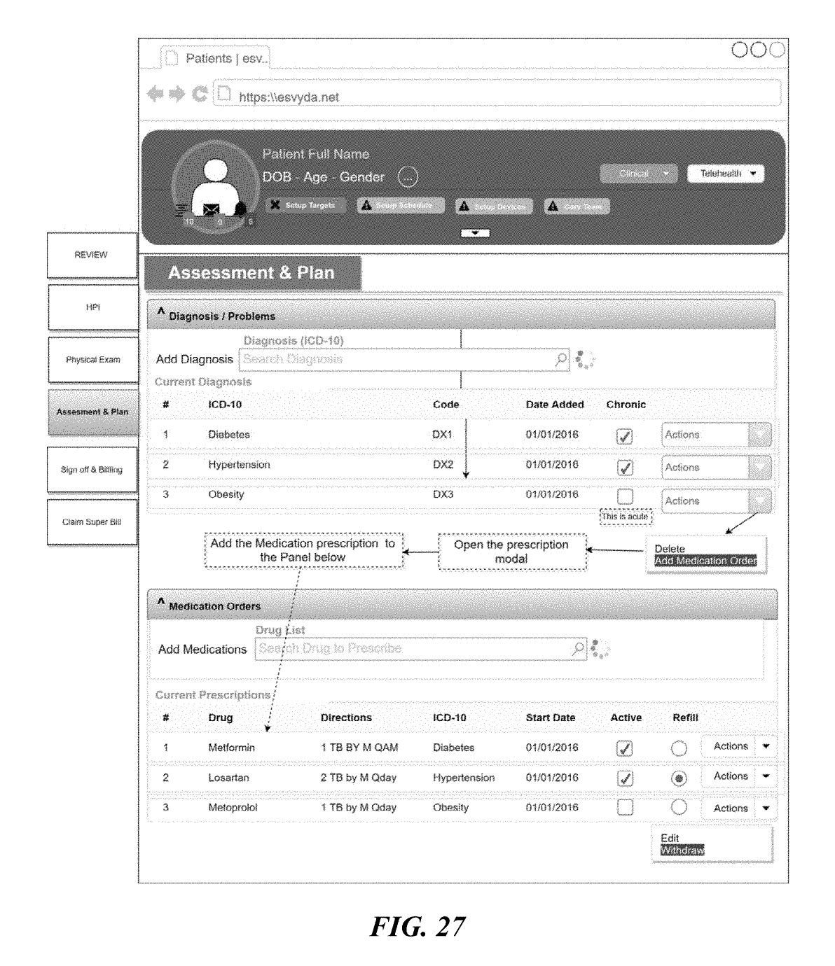

FIG. 27 illustrates an example of a medication order consultation module interface for the evaluation of the diagnosis of users, consistent with various embodiments.

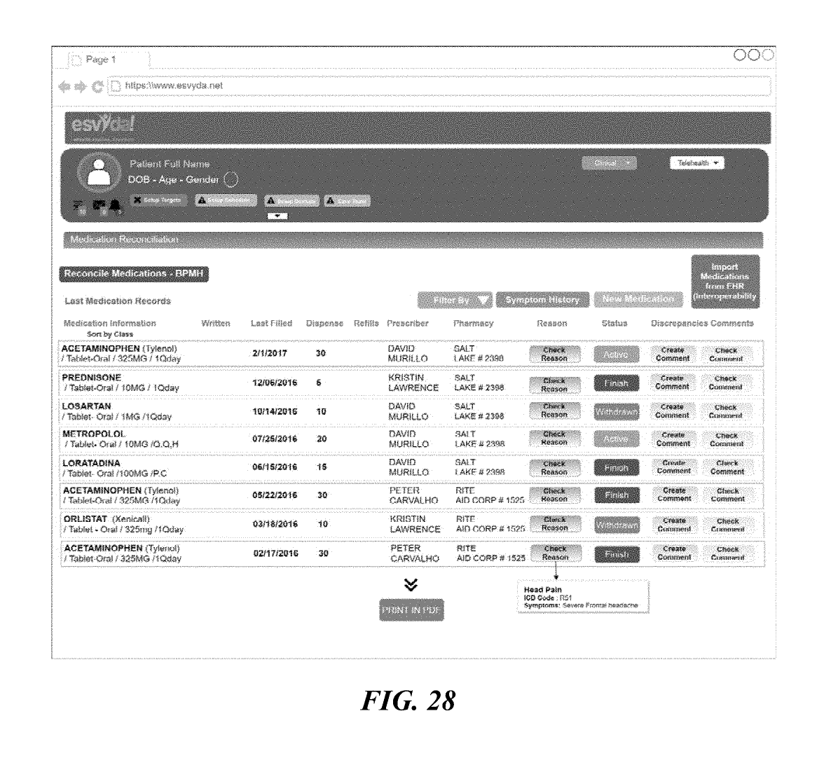

FIG. 28 illustrates an example medication reconciliation module interface of a medication history of user, consistent with various embodiments.

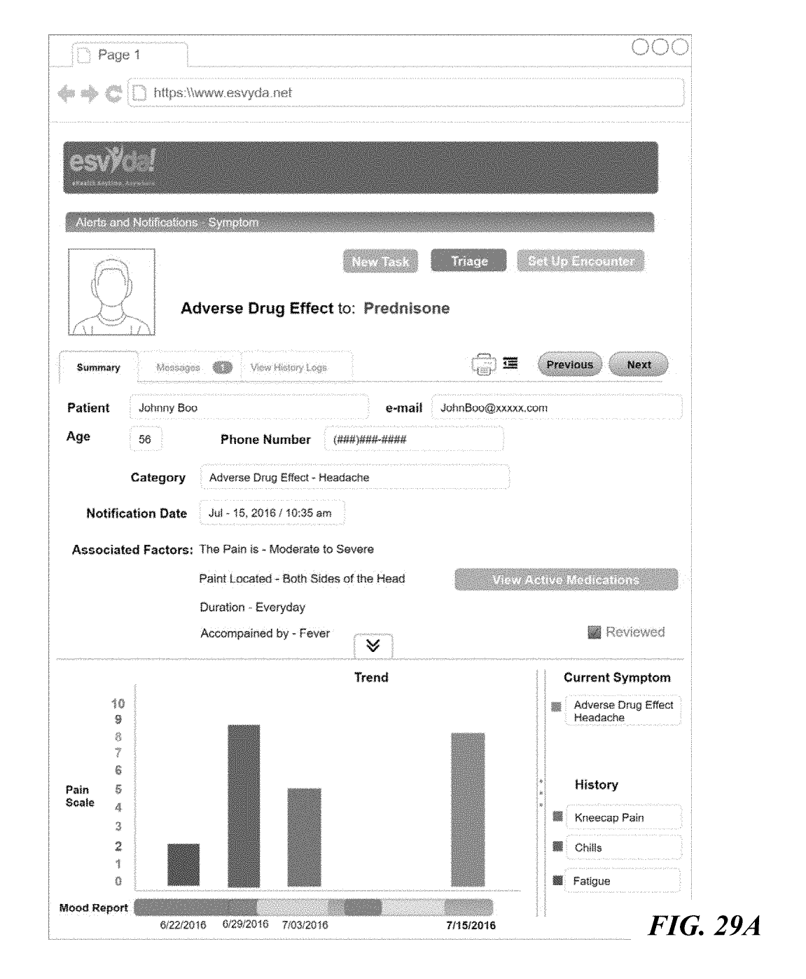

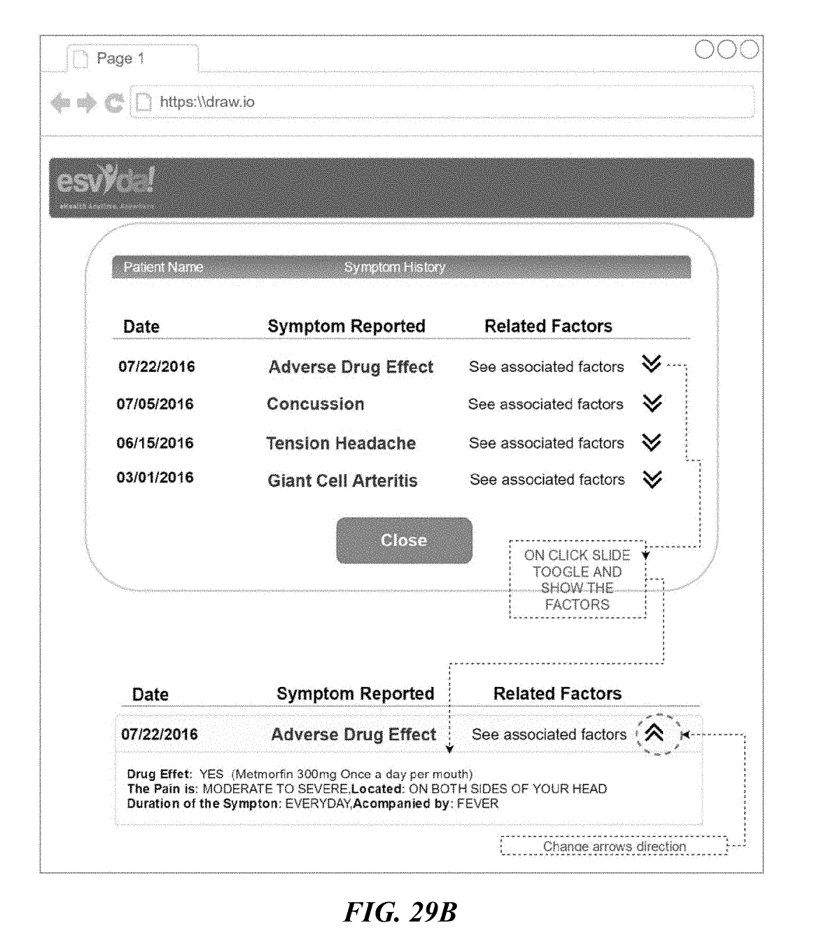

FIGS. 29A-29B illustrate examples of alerts and notifications module interfaces that notifies users of generated events generated, consistent with various embodiments.

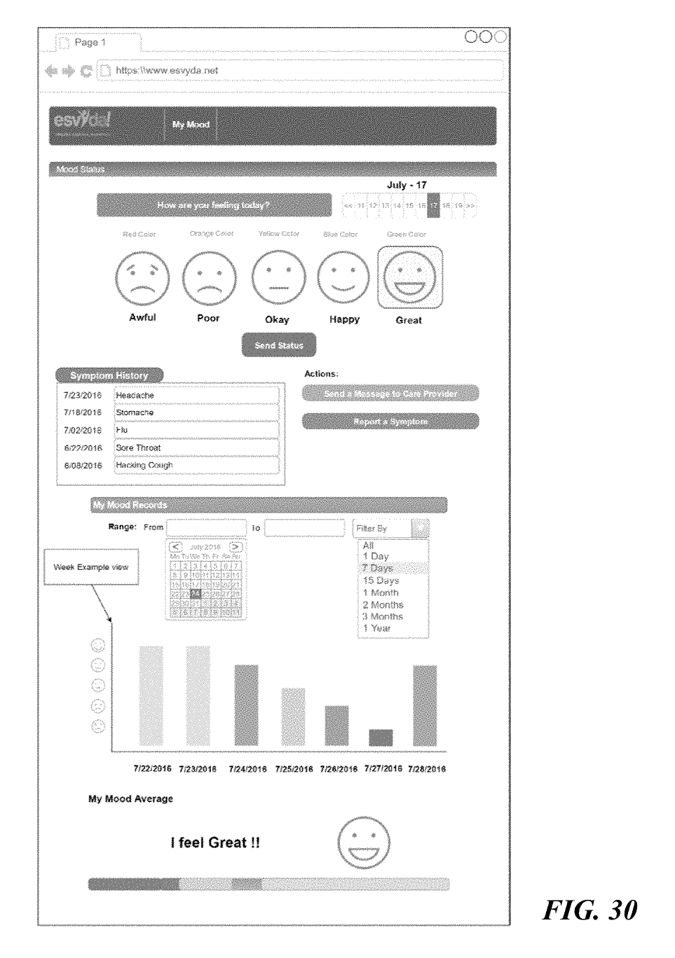

FIG. 30 illustrates an example of a mood module interface that allows users to report current status against medications prescribed, consistent with various embodiments.

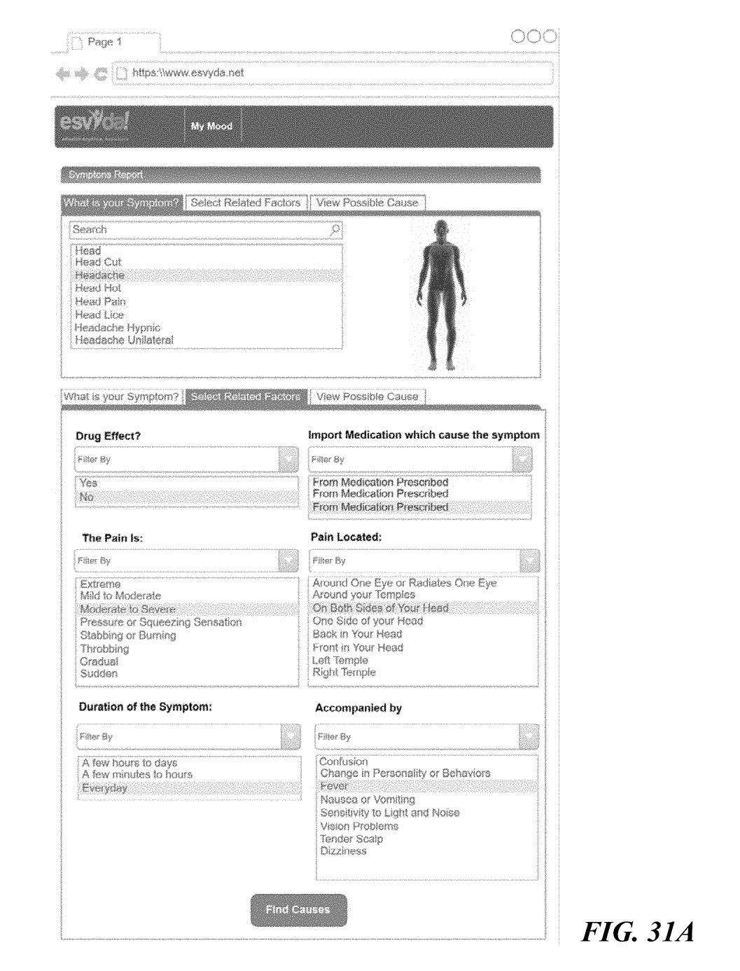

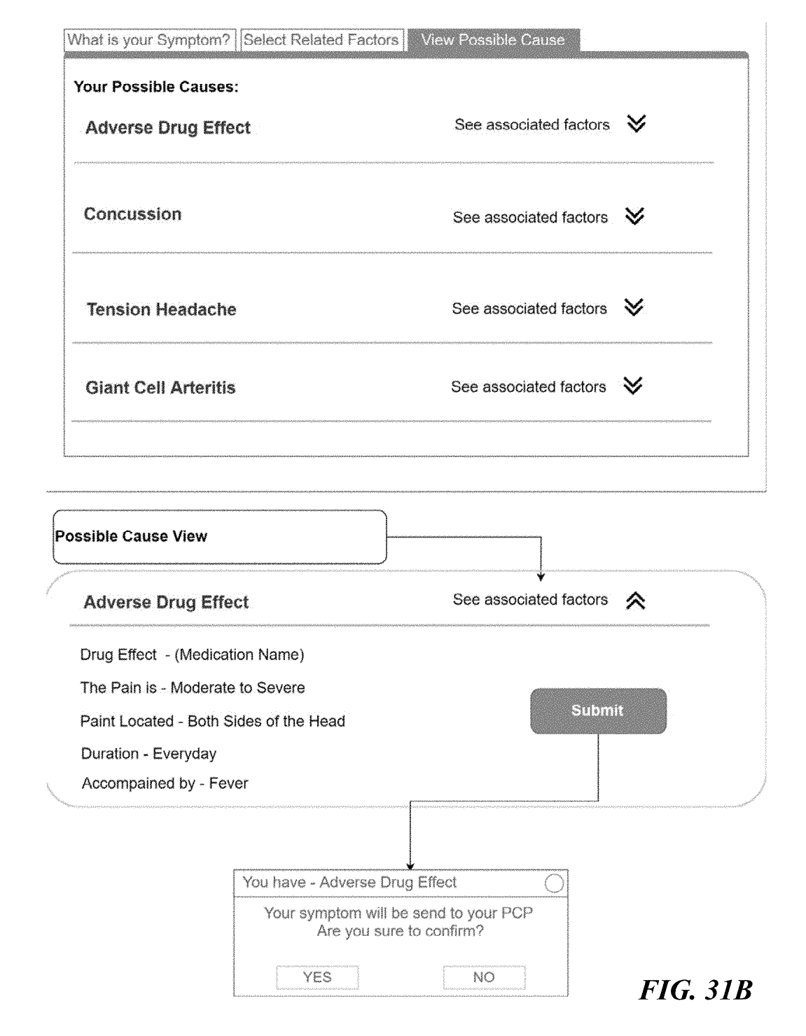

FIGS. 31A-31B illustrate examples of events report module interfaces that allow users or care providers to report any medical event related to the care plan, consistent with various embodiments.

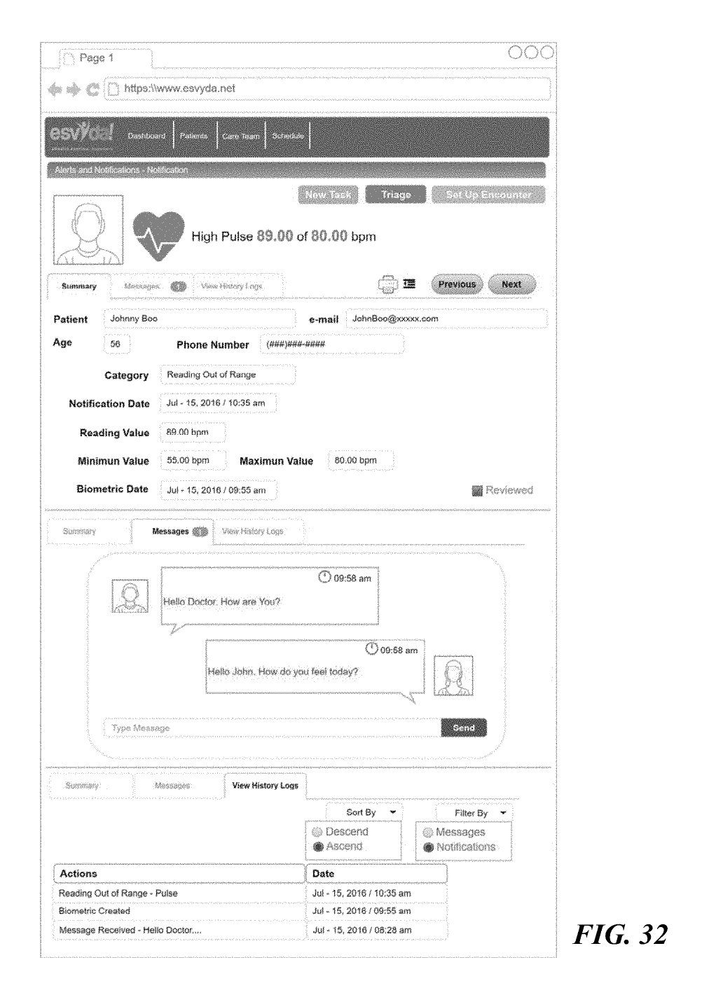

FIG. 32 illustrates an example alerts and notifications module interface with the vital sign alerts generated by users engaged in a care plan, consistent with various embodiments.

FIG. 33 illustrates an example of an engagement module interface that shows the medication adherence metrics during the progression of the care plan assigned to a user, consistent with various embodiments.

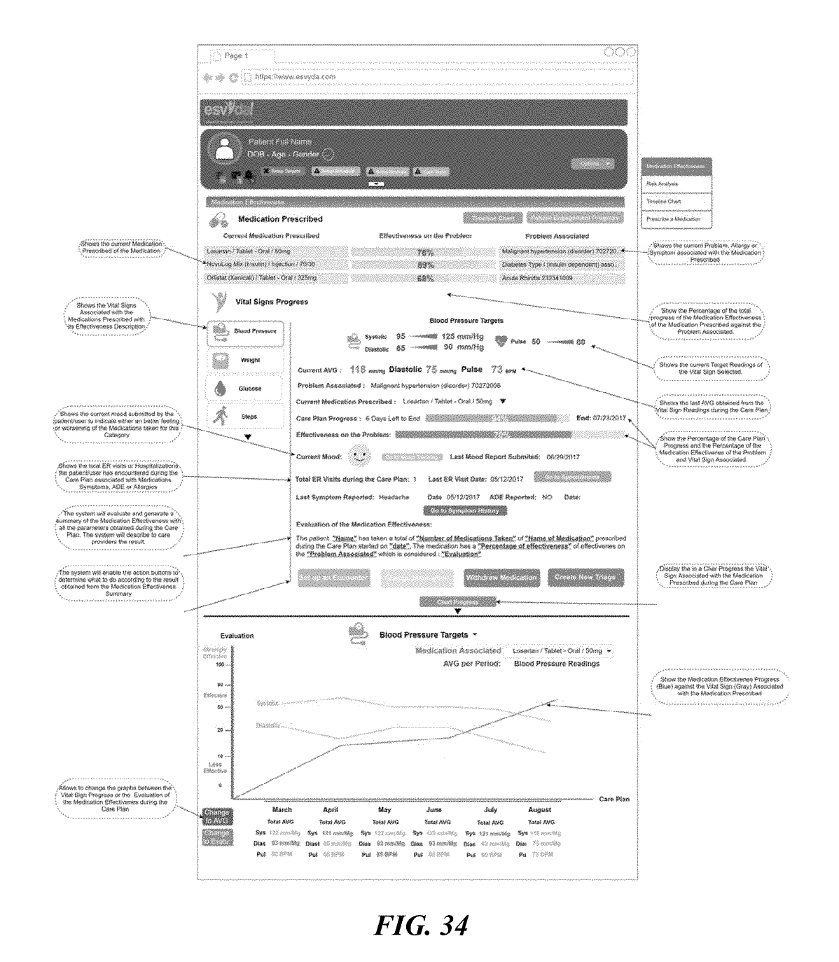

FIG. 34 illustrates an example care plan program module interface that shows the effectiveness based on medical parameters and medical events, consistent with various embodiments.

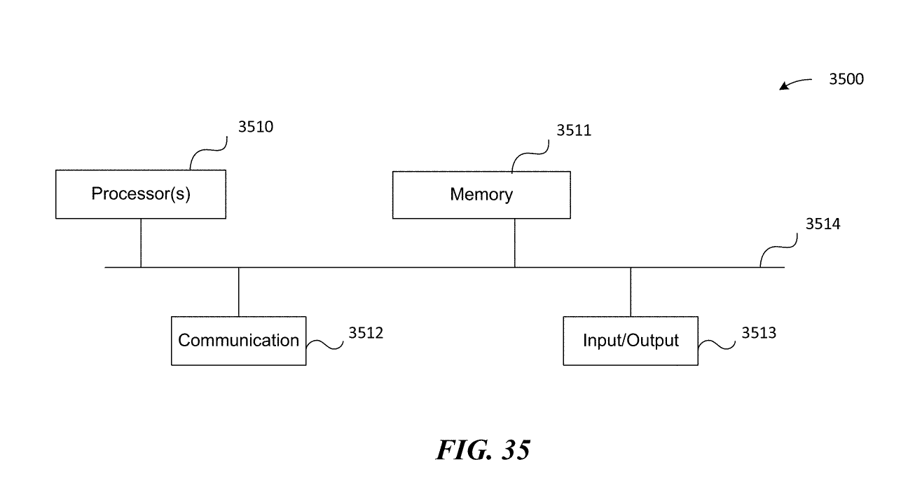

FIG. 35 is a block diagram illustrating an example of a processing system in which at least some operations described herein can be implemented, consistent with various embodiments. Processing device 3500 can represent any of the devices described above, e.g., a telemonitoring and analysis system, a mobile device, a computing device, etc. Any of these systems can include two or more processing devices, as is represented in FIG. 35, which can be coupled to each other via a network or multiple networks.

In the illustrated embodiment, the processing system 3500 includes one or more processors 3510, memory 3511, a communication device 3512, and one or more input/output (I/O) devices 3513, all coupled to each other through an interconnect 3514. The interconnect 3514 may be or include one or more conductive traces, buses, point-to-point connections, controllers, adapters and/or other conventional connection devices. The processor(s) 3510 may be or include, for example, one or more general-purpose programmable microprocessors, microcontrollers, application specific integrated circuits (ASICs), programmable gate arrays, or the like, or any combination of such devices. The processor(s) 3510 control the overall operation of the processing device 3500. Memory 3511 may be or include one or more physical storage devices, which may be in the form of random access memory (RAM), read-only memory (ROM) (which may be erasable and programmable), flash memory, miniature hard disk drive, or other suitable type of storage device, or any combination of such devices. Memory 3511 may store data and instructions that configure the processor(s) 3510 to execute operations in accordance with the techniques described above. The communication device 3512 may be or include, for example, an Ethernet adapter, cable modem, Wi-Fi adapter, cellular transceiver, Zigbee transceiver, Bluetooth transceiver, or the like, or any combination thereof. Depending on the specific nature and purpose of the processing device 3500, the I/O devices 3513 can include various devices, e.g., a display (which may be a touch screen display), audio speaker, keyboard, mouse or other pointing device, microphone, camera, etc.

Unless contrary to physical possibility, it is envisioned that (i) the methods/steps described above may be performed in any sequence and/or in any combination, and that (ii) the components of respective embodiments may be combined in any manner.

The techniques introduced above can be implemented by programmable circuitry programmed/configured by software and/or firmware, or entirely by special-purpose circuitry, or by any combination of such forms. Such special-purpose circuitry (if any) can be in the form of, for example, one or more application-specific integrated circuits (ASICs), programmable logic devices (PLDs), field-programmable gate arrays (FPGAs), etc.

Software or firmware to implement the techniques introduced here may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc.

Note that any and all of the embodiments described above can be combined with each other, except to the extent that it may be stated otherwise above or to the extent that any such embodiments might be mutually exclusive in function and/or structure.

Although the present technology has been described with reference to specific exemplary embodiments, it will be recognized that the technology is not limited to the embodiments described, but can be practiced with modification and alteration within the spirit and scope of the appended claims. Accordingly, the specification and drawings are to be regarded in an illustrative sense rather than a restrictive sense.

We claim:

1. A method for determining effectiveness of a care plan, the method comprising:
accessing, by a telemonitoring and analysis system, a care plan of a patient,
wherein the care plan includes a plurality of scheduled care plan events,
wherein the scheduled care plan events are associated with medical parameters;
receiving, from one or more devices, medical parameter data associated with the medical parameters for each of the scheduled care plan events;
determining, by the telemonitoring and analysis system, an adherence level for each of the medical parameters, wherein determining the adherence level comprises:
for each of the medical parameters, comparing the medical parameter data for each of the scheduled care plan events with expected medical parameter data for each of the scheduled care plan events,
classifying the medical parameter data into scoring classes based on the comparison,
assigning a weighted value to the medical parameter data for each of the scheduled care plan events, wherein the weighted value is based on the scoring class, and
averaging the weighted value of the medical parameter data for each of the medical parameters to determine the adherence level for each of the medical parameters;
receiving medical event data relating to medical events occurring during the care plan;
generating, by the telemonitoring and analysis system, a medical event score, wherein generating the medical event score comprises:
categorizing the medical event data into medical event subcategories, assigning a weight to each of the subcategories,
determining a subcategory score for each of the medical event subcategories based on the weight and the medical event data, and
combining the subcategory scores to generate the medical event score;
normalizing the adherence level for each of the medical parameters and the medical event score; and
calculating, by the telemonitoring and analysis system, an effectiveness of the care plan based at least in part on the normalized adherence levels and the normalized medical event score.

2. The method of claim 1, wherein the medical parameters include medication data and biometric data, wherein each of the medial parameters is associated with more than one adherence level.

3. The method of claim 2, wherein the method further comprises:
simultaneously receiving the medication data from a mobile device and a drug dispenser device;
comparing the medication data received from the drug dispenser device with the medication data received from the mobile device; and
selecting the medication data based on a reliability of a source of the medication data when the medication data received from the mobile device is different from the medication data received from the drug dispenser device.

4. The method of claim 1, wherein the method further comprises sending an alert to a care provider when the medical parameter data indicates an overdose of medication.

5. The method of claim 1, wherein the medical event subcategories include adverse reactions, visits to an emergency room or hospital, symptoms, allergies, Adverse Drug Effects, and Adverse Drug Reactions.

6. The method of claim 1, wherein the method further comprises:
generating, by the telemonitoring and analysis system, scores for external sources, wherein the external sources include events, mood information, survey information, and perceptions from care providers.

7. The method of claim 6, wherein the method further comprises normalizing the scores for the external sources, wherein calculating the effectiveness of the care plan is further based on the normalized scores for the external sources.

8. The method of claim 7, wherein the weight of the adherence levels and the medical event scores are modified with a correction factor, wherein the correction factor is based on the external sources, expert considerations and analysis of an average, variance and covariance of weights assigned to other individuals with similar demographic characteristics, wherein the demographic characteristics include one or more of age, gender, ethnic origin, race and economic status.

9. The method of claim 7, wherein the correction factor is further based on sums of the square of deviation and data normalization.

10. The method of claim 1, wherein the weights of the subcategories are determined by one or more experts and a level of risk of the patient.

11. A telemonitoring and analysis system comprising:
a processor;
a storage device coupled to the processor;
a networking interface coupled to the processor; and
a memory coupled to the processor and storing instructions which, when executed by the processor, cause the telemonitoring and analysis system to perform operations including:
accessing a care plan of a patient,
wherein the care plan includes a plurality of scheduled care plan events,
wherein the scheduled care plan events are associated with medical parameters,
receiving, from one or more devices, medical parameter data associated with the medical parameters for each of the scheduled care plan events,
determining an adherence level for each of the medical parameters, wherein determining the adherence level comprises:
for each of the medical parameters, comparing the medical parameter data for each of the scheduled care plan events with expected medical parameter data for each of the scheduled care plan events,
classifying the medical parameter data into scoring classes based on the comparison,
assigning a weighted value to the medical parameter data for each of the scheduled care plan events, wherein the weighted value is based on the scoring class, and
averaging the weighted value of the medical parameter data for each of the medical parameters to determine the adherence level for each of the medical parameters,
receiving medical event data relating to medical events occurring during the care plan,
generating a medical event score, wherein generating the medical event score comprises:
categorizing the medical event data into medical event subcategories, assigning a weight to each of the subcategories,
determining a subcategory score for each of the medical event subcategories based on the weight and the medical event data, and
combining the subcategory scores to generate the medical event score,
normalizing the adherence level for each of the medical parameters and the medical event score, and
calculating an effectiveness of the care plan based at least in part on the normalized adherence levels and the normalized medical event score.

12. The system of claim 11, wherein the medical parameters include medication data and biometric data, wherein each of the medial parameters is associated with more than one adherence level.

13. The system of claim 12, wherein the operations further include:
simultaneously receiving the medication data from a mobile device and a drug dispenser device;
comparing the medication data received from the drug dispenser device with the medication data received from the mobile device; and
selecting the medication data based on a reliability of a source of the medication data when the medication data received from the mobile device is different from the medication data received from the drug dispenser device.

14. The system of claim 11, wherein the operations further comprise sending an alert to a care provider when the medical parameter data indicates an overdose of medication.

15. The system of claim 11, wherein the medical event subcategories include adverse reactions, visits to an emergency room or hospital, symptoms, allergies, Adverse Drug Effects, and Adverse Drug Reactions.

16. The system of claim 11, wherein the operations further comprise:
generating scores for external sources, wherein the external sources include events, mood information, survey information, and perceptions from care providers.

17. The system of claim 16, wherein the operations further comprise normalizing the scores for the external sources, wherein calculating the effectiveness of the care plan is further based on the normalized scores for the external sources, wherein the weights of the subcategories are determined by one or more experts and a level of risk of the patient.

18. The system of claim 17, wherein the weight of the adherence levels and the medical event scores are modified with a correction factor, wherein the correction factor is based on the external sources, expert considerations and analysis of an average, variance and covariance of weights assigned to other individuals with similar demographic characteristics, wherein the demographic characteristics include one or more of age, gender, ethnic origin, race and economic status.

19. The system of claim 17, wherein the correction factor is further based on sums of the square of deviation and data normalization.

20. At least one non-transitory computer-readable medium comprising a set of instructions associated with a telemonitoring and analysis system that, when executed by one or more processors, cause the telemonitoring and analysis system to perform operations of:
accessing a care plan of a patient,
wherein the care plan includes a plurality of scheduled care plan events,
wherein the scheduled care plan events are associated with medical parameters;
receiving, from one or more devices, medical parameter data associated with the medical parameters for each of the scheduled care plan events;
determining, by the telemonitoring and analysis system, an adherence level for each of the medical parameters, wherein determining the adherence level comprises:
for each of the medical parameters, comparing the medical parameter data for each of the scheduled care plan events with expected medical parameter data for each of the scheduled care plan events,
classifying the medical parameter data into scoring classes based on the comparison,
assigning a weighted value to the medical parameter data for each of the scheduled care plan events, wherein the weighted value is based on the scoring class, and
averaging the weighted value of the medical parameter data for each of the medical parameters to determine the adherence level for each of the medical parameters;
receiving medical event data relating to medical events occurring during the care plan;
generating a medical event score, wherein generating the medical event score comprises:
categorizing the medical event data into medical event subcategories,
assigning a weight to each of the subcategories,
determining a subcategory score for each of the medical event subcategories based on the weight and the medical event data, and
combining the subcategory scores to generate the medical event score;
normalizing the adherence level for each of the medical parameters and the medical event score; and
calculating an effectiveness of the care plan based at least in part on the normalized adherence levels and the normalized medical event score.
